(19) United States
(12) Patent Application Publication
(10) Pub. No.: US 2003/0064527 A1
(43) Pub. Date: Apr. 3, 2003

(54) Proteomic differential display

(75) Inventors: David M. Lubman, Ann Arbor, MI (US); Maureen Kachman, Ann Arbor, MI (US); Stephen J. Parus, Ann Arbor, MI (US)

Correspondence Address: MEDLEN & CARROLL, LLP, Suite 350, 101 Howard Street, San Francisco, CA, 94105, US

(73) Assignee: The Regents of the University of Michigan, Ann Arbor, MI (US)

(21) Appl. No.: 10/133,896

(22) Filed: Apr. 26, 2002

Related U.S. Application Data
(63) Continuation-in-part of application No. 09/778,548, filed on Feb. 7, 2001.
(60) Provisional application No. 60/288,170, filed on May 2, 2001.

(51) Int. Cl. G01N 1/00

(52) U.S. Cl. 436/174

(57) ABSTRACT

The present invention relates to multi-phase protein separation methods capable of resolving and characterizing large numbers of cellular proteins, including methods for efficiently facilitating the transfer of protein samples between separation phases. In particular, the present invention provides systems and methods for the differential display of protein samples from multiple cell types. The present invention thus provides improved methods for the analysis of multiple samples containing large numbers of proteins.

The present appliction is a continuation in part of U.S. patent application Ser. No. 09/778,548, filed Feb. 7, 2001 and claims priority to U.S. Provisional Patent application Ser. No. 60/288,170 filed May 2, 2001.

The present invention was made, in part, with government funding under National Institutes of Health under grant No. R01GM49500 and the National Science Foundation grant No. DBI-9987220. The government has certain rights in this invention.

### FIELD OF THE INVENTION

The present invention relates to multi-phase protein separation methods capable of resolving and characterizing large numbers of cellular proteins, including methods for efficiently facilitating the transfer of protein samples between separation phases. In particular, the present invention provides systems and methods for the differential display of protein samples from multiple cell types.

### BACKGROUND OF THE INVENTION

As the nucleic acid sequences of a number of genomes, including the human genome, become available, there is an increasing need to interpret this wealth of information. While the availability of nucleic acid sequence allows for the prediction and identification of genes, it does not explain the expression patterns of the proteins produced from these genes. The genome does not describe the dynamic processes on the protein level. For example, the identity of genes and the level of gene expression does not represent the amount of active protein in a cell nor does the gene sequence describe post-translational modifications that are essential for the function and activity of proteins. Thus, in parallel with the genome projects there has begun an attempt to understand the proteome (i.e., the quantitative protein expression pattern of a genome under defined conditions) of various cells, tissues, and species. Proteome research seeks to identify targets for drug discovery and development and provide information for diagnostics (e.g., tumor markers).

An important area of research is the study of the protein content of cells (i.e., the identity of and amount of expressed proteins in a cell). This field requires methods that can separate out large numbers of proteins and can do so quantitatively so that changes in expression or structure of proteins can be detected. The method generally used to achieve such cellular protein separations is 2-D PAGE. This method is capable of resolving hundreds of proteins based upon pI in one dimension and protein size in the second dimension. The proteins separated by this method are visualized using a staining method that can generally be quantified. The result is a 2-dimensional image where the protein map is based on pI and approximate molecular weight. By the use of computer based image analysis techniques, one can search for proteins that are differentially expressed in various cell lines. These methods are used to monitor changes in protein expression that are linked to conditions such as cell transformation and cancer progression, cell aging, the response of cells to environmental insult, and the response of cells to pharmaceutical agents. Once changes in protein expression have been identified, then one can further analyze target proteins to determine their identity and whether they have been altered from their expected structure by sequence changes or post-translational modifications.

Although 2-D PAGE is still widely used for protein analysis, the method has several limitations including the fact that it is labor intensive, time consuming, difficult to automate and often not readily reproducible. In addition, quantitation, especially in differential expression experiments, is often difficult and limited in dynamic range. Also, while the 2-D gel does produce an image of the proteins in the cell, the mass determination is often only accurate to 5-10%, and the method is difficult to interface to mass spectrometric techniques for further analysis.

Another limitation of 2-D PAGE is the amount of protein loaded per gel which is generally below 250 μg. The amount of protein in any given spot may therefore be too low for further analysis. For Coomassie brilliant blue (CBB) stained gels the limit of detection is 100 ng per spot while for silver stained gels the limit of detection is 1-10 ng. Furthermore, proteins that have been isolated in 2-D gels are embedded inside the gel structure and are not free in solution, thus making it difficult to extract the protein for further analysis. Because of these limitations, the art is in need of protein mapping methods that are efficient, automated, and have broader resolution capabilities than presently available technologies.

### SUMMARY OF THE INVENTION

The present invention relates to multi-phase protein separation methods capable of resolving and characterizing large numbers of cellular proteins, including methods for efficiently facilitating the transfer of protein samples between separation phases. In particular, the present invention provides systems and methods for the differential display of protein samples from multiple cell types.

For example, in some embodiments, the present invention provides a method for summing mass spectrum data, comprising providing a mass spectrum generated from a separated protein sample; identifying regions of the mass spectrum that contain mass data for a first protein; and summing the regions of the mass spectrum to generate summed mass spectrum. In some embodiments, the separated protein sample comprises a separated cell lysate. In some embodiments, the separated cell lysate is separated in a first and second separation dimension. The present invention is not limited to separation in any particular first and second dimensions. For example, in some embodiments, the first separation dimension represents protein isoelectric point and the second separation dimension represents protein hydrophobicity. In some embodiments, the cell lysate is further separated based on molecular weight and abundance. In some embodiments, the method further comprises displaying the summed mass spectra. In some embodiments, the summed mass spectra are displayed as a 2-dimensional map. In some embodiments, the 2-dimensional map comprises a first axis representing isoelectric point and a second axis representing mass. In some embodiments, the 2-dimensional map further displays protein abundance of proteins represented in the 2-dimensional plot. In some embodiments, proteins are represented as bands in the 2-dimensional map and the intensity of the bands represents relative protein abundance of the bands. In some embodiments, the 2-dimensional map is displayed on a computer video screen. In some embodiments, the summing of step is performed manually. In other embodiments, the summing is performed by a computer processor.

The present invention additionally provides a method for displaying proteins comprising providing a first 2-dimensional protein map representing a first sample comprising a plurality of proteins; a second 2-dimensional protein map representing a second sample comprising a plurality of proteins; and a computer system comprising display software and a display screen; and subtracting the second 2-dimensional protein map from the first two dimension protein map with the display software to generate a differential display map; and displaying the differential display map on the display screen. In some embodiments, the differential display map represents differences in protein composition between the first and second 2-dimensional protein maps as bands, and wherein each band represents one protein. In some embodiments, the bands comprise bands of two different colors, and each of the two different colors corresponds to proteins from each of the first and second samples. In other embodiments, the bands comprise bands of two different color gradients, and each of the two different color gradients correspond to proteins from each of the first and second samples. In some embodiments, the differences in protein composition represent differences in abundance of the same protein displayed in each of the first and second 2-dimensional protein maps. In other embodiments, the differences in protein composition represent the presence or absence proteins in each of the first and second 2-dimensional protein maps. In still further embodiments, the bands comprise bands of four different colors, wherein two of the four colors each correspond to protein from each of the first and second samples, and wherein two of the four colors each represent bands where one of the cell lines is lacking a particular protein.

In some embodiments, the first and second 2-dimensional protein maps represent separation of the first and second proteins samples in a first dimension and a second dimension. In some embodiments, the first dimension is isoelectric point and the second dimension is hydrophobicity. In some embodiments, the first and second 2-dimensional protein maps further represent characterization of protein mass and abundance.

In some embodiments, the differential display map further comprises hyperlinks. In some embodiments, the hyperlinks are links to information corresponding to proteins represented by the bands of the differential display image. The hyperlinks may link to any relevant information corresponding to the proteins of the differential display map, including but not limited to, protein identity, molecular weight, relative abundance, isolectric point, and hydrophobicity.

The present invention also provides a system for displaying protein differential display maps, comprising: a protein differential display map displayed on a display screen; and a plurality of hyperlinks displayed on the display screen, wherein the hyperlinks correspond to individual regions of the protein differential display map, and wherein the hyperlinks are links to information corresponding to the regions. In some embodiments, the protein differential display map represents differences in protein composition between first and second 2-dimensional protein plots. In some embodiments, the differences in protein composition are represented as bands, and each band represents one protein. In some embodiments, each of the regions is a band corresponding to one protein. The hyperlinks may link to any relevant information corresponding to the proteins of the differential display map, including but not limited to, protein identity, molecular weight, relative abundance, isolectric point, and hydrophobicity.

### DESCRIPTION OF THE FIGURES

FIG. 1 shows an example 2-D protein display using Isoelectric Focusing Non-Porous Reverse Phase HPLC (IEF-NP RP HPLC) separation of human erythroleukemia cell lysate proteins in one embodiment of the present invention.

FIG. 2 shows a zoom area of a portion of the display in FIG. 1 (pI=4.2 to 7.2 and tR=6.0 to 9.0) (right panel showing banding patterns) and a corresponding example of linked HPLC data (left panel showing peaks).

FIG. 3 shows a quantification of rotofor fractions in one embodiment of the present invention.

FIG. 4 shows NP RP HPLC separation from a Rotofor fraction of HEL cell lysate in one embodiment of the present invention.

FIGS. 5A and 5B show short (5A) and long (5B) NP RP HPLC separation gradient times for a rotofor fraction of HEL cell lysate in one embodiment of the present invention.

FIG. 6 shows an example of Coomassie blue stained 2-D PAGE separation of HEL cell lysate proteins.

FIG. 7 shows a direct side-by-side comparison of IEF-NP RP HPLC (four lanes on the left) with 1-D SDS PAGE (four lane on the right) for several Rotofor fractions in certain embodiments of the present invention.

FIGS. 8A and 8B show MALDI-TOF MS tryptic peptide mass maps for α-enolase isolated by IEF-NP RP HPLC (8A) and by 2-D PAGE (8B).

FIG. 9 shows a 2D protein image of Isoelectric Focusing-Non-porous RP HPLC-ESI oa TOF/MS (IEF-NPS RP HPLC-ESI oa TOF/MS) separation of human erythroleukemia cell lysate proteins.

FIG. 10 shows a zoom of the 2D protein image from FIG. 9 of 35 kDa to 52 kDa mass range.

FIG. 11A and 11B show actin multiply charged umbrella with MaxEnt deconvoluted molecular weight mass spectrum. The umbrella for beta and gamma actin is shown in FIG. 11A, each form of actin being labeled with the charge state. FIG. 11B shows the resulting molecular weight mass spectrum for actin where the two forms of actin are separated.

FIG. 12 shows combined protein molecular weight mass spectrum from a Rotofor fraction shown in traditional peak format.

FIG. 13 shows a zoom of 2D protein image from FIG. 9 of 5 kDa to 40 kDa mass range.

FIG. 14 shows a chromatofocusing profile of MCF-10A whole cell lysate.

FIGS. 15A, B, and C show NP-RP-HPCL-ESI-oaTOF TIC (total ion count) profile of three sample fractions identified in FIG. 14.

FIG. 16 shows an integrated and deconvoluted TIC profile of the three sample fractions from FIG. 15, as generated with MaxEnt1 software.

FIG. 17 shows the anion exchange profile of Siberian Permafrost whole cell lysate of sample 23-9-25.

FIGS. 18A and 18B show the NP-RP-HPLC-ESI-oaTOF TIC profile of two fractions from FIG. 17.

FIG. 19 shows a single mass spectrum from a IEF/RP NPS/ESI-oaTOF/MS separation.

FIG. 20 shows a TIC from a IEF/RP NPS/ESI-oa TOF/MS separation.

FIG. 21 shows a deconvoluted mass spectrum showing the protein molecular weight.

FIG. 22 shows a 2-dimensional plot of pI vs. mass for nine Rotofor fractions from a cancer cell line.

FIG. 23 shows a differential display image of the 10-35 kDa region of a single pI fraction from two cell types. The 2-dimensional map for the ES2 ovarian cancer cell line is on the left and the 2-dimensional map for normal ovarian epithelial cells is on the right. The middle band shows the differences between the two cell types.

FIG. 24 shows a Table of proteins identified in ES2 and OSE with quantification and hydrophobicity comparison.

FIG. 25 shows 2-Dimensional mass maps of MW versus pI comparing the ES2 cell line to the OSE cell line for Rotofor fraction nos. (a) 6, (b) 7, and (c) 14. The names of proteins identified by MALDI-TOFMS peptide mapping are listed with the corresponding MW bands according to the labeling scheme of FIG. 23.

FIG. 26 shows NPS RP-HPLC chromatograms of Rotofor fraction 7 for FIG. 26(a) ES2 cell line and FIG. 26(b) OSE cell line with detection by UV absorption at 214 nm. The names of proteins identified by liquid fraction collection, tryptic digestion, and MALDI-TOFMS peptide mapping are listed with the corresponding chromatographic peak.

FIG. 27 shows a Table of purported proteins not identified by MALDI but present in Fraction 6 in Both ES2 and OSE.

FIG. 28 shows a comparison of the mass maps for fractions 6 and 7 between the OSE cell lines and the ES2 cell lines.

### GENERAL DESCRIPTION OF THE INVENTION

The present invention relates to multi-phase protein separation methods capable of resolving large numbers of cellular proteins, including methods for efficiently facilitating the transfer of protein samples between separation phases. The methods of the present invention provide protein profile maps for imaging and comparing protein expression patterns. The present invention provides alternatives to traditional 2-D gel separation methods for the screening of protein profiles. Many limitations of traditional 2-D PAGE arise from its use of the gel as the separation media. The present invention provides alternative media for the separation that offer significant advantages over 2-D PAGE techniques. For example, in some embodiments, the present invention provides methods that use two dimensional separations, where the second dimensional separation occurs in the liquid phase, rather than 2-D PAGE techniques where the final separation occurs in gel.

The present invention provides systems and methods for protein separation and mapping that are highly efficient, amenable to automation, and provide detailed resolution. For example, in some methods of the present invention, proteins are separated according to their pI, using isoelectric focusing (IEF) (e.g., in the Rotofor); according to their hydrophobicity using non-porous reverse phase HPLC (NPS RP HPLC); and according to mass using ESI oa TOF/MS or other mass spectrometry techniques. The present invention further provides novel techniques for eluting proteins from a separation apparatus (e.g., the first phase separation apparatus). For example, in one embodiment of the present invention, the proteins eluted from the first dimension are “peeled off” from the column according to their pH, either one pH unit or fraction thereof, at a time. In some embodiments, these focused liquid fractions are then separated according to their hydrophobicity and size (or other desired properties) in the second dimension. Liquid fractions from, for example, NP-RP-HPLC can be conveniently analyzed directly on-line using mass spectrometry (e.g., ESI-oaTOF) to obtain their molecular weight and relative abundance, which provides a third dimension. As a result, a virtual 2-D protein image is created and is analogous to a 2-D gel image.

Experiments conducted during the development of the present invention have demonstrated that these methods are capable of separating large numbers of proteins. The 2-D image of these proteins, analogous to that of a 2-D gel, can be generated for the purpose of observing distinctive patterns from a particular cell line. This protein pattern provides relative quantitative information, high mass resolution and high accuracy pI and mass values. Given that the intensity, mass and pI values are reproducible, one can study differential expression of proteins where the resulting 2-D images from different cells, tissues, or samples can be quantitatively compared to identify points of interest. Furthermore, automation and speed of analysis are greatly facilitated given that the proteins remain in the liquid phase throughout the separation. The method, abbreviated IEF-NPS RP HPLC-ESI oa TOF/MS is shown to be a viable alternative for the separation of complex protein mixtures and the generation of high-resolution 2-D images of cellular protein expression.

In some embodiments of the present invention, proteins are separated in a first dimension using any of a large number of protein separation techniques including, but not limited to, ion exclusion, ion exchange, normal/reversed phase partition, size exclusion, ligand exchange, liquid/gel phase isoelectric focusing, and adsorption chromatography. In some preferred embodiments of the present invention, the first dimension is a liquid phase separation method. The sample from the first separation is passed through a second dimension separation. In preferred embodiments of the present invention, the second dimension separation is conducted in liquid phase. The products from the second dimension separation are then characterized. For example, in preferred embodiments, the products of the second separation step are detected and displayed in a 2-D format based on the physical properties of the proteins that were distinguished in the first and second separation steps (e.g., under conditions such that the first and the second physical properties are revealed for at least a portion of the proteins). The products may be further analyzed, for example, by mass spectrometry to determine the mass and/or identity of the products or a subset of the products. In these embodiments, a three dimensional characterization can be applied (i.e., based on the physical properties of the first two separation steps and the mass spectrometry data). It is contemplated that other protein processing steps can be conducted at any stage of the process.

In certain embodiments of the present invention, the steps are combined in an automated system. In preferred embodiments, each of the steps is automated. For example, the present invention provides a system that includes each of the separation and detection elements in operable combination so that a protein sample is applied to the system and the user receives expression map displays or other desired data output. To achieve automation, in preferred embodiments, the products of each step should be compatible with the subsequent step or steps.

In one illustrative embodiment of the present invention proteins are separated according to their pI, using isoelectric focusing (IEF) in a Rotofor and according to their hydrophobicity and molecular weight using NP RP HPLC. This combined separation method is abbreviated IEF-NP RP HPLC. When coupled with mass spectrometry (MS) this technique becomes three-dimensional and allows for the creation of a protein map that tells the pI and the molecular weight of the proteins in question. This information can be plotted in an image that also depicts protein abundance. The end result is a high-resolution image showing a complex pattern of proteins separated by pI and molecular weight and indicating relative protein abundances. This image can be used to determine how the proteins in a given cell line or tissue may change due to some disease state, pharmaceutical treatment, natural or induced differentiation, or change in environmental conditions. The image allows the observer to determine changes in pI, molecular weight, and abundance of any protein in the image. When interfaced to MS the identity of any target protein may also be obtained via enzymatic digests and peptide mass map analyses. In addition, this technique has the advantage of very high loadability (e.g., 1 gram) such that the lower abundance proteins may be detected.

In traditional 2-D PAGE separation and display techniques, the second phase separation is conducted in a gel (i.e., not a liquid phase) and the proteins are separated and detected by differences in molecular weight. In contrast, in some embodiments of the present invention, the second phase separation is conducted in liquid phase. The products of the second phase separation techniques of the present invention are much more amenable to further characterization and to interpretation of data produced from the second phase. For example, in some embodiments of the present invention, the second phase is conducted using HPLC where the separated protein products are readily detected as peak fractions and interpreted and displayed in two dimensions by a computer based on the physical properties of the first and second separation steps. The products of HPLC separation, being in the liquid phase, are readily used in further detection steps (e.g., mass spectrometry). The methods of the present invention, as compared to traditional 2-D PAGE, allow more sample to be analyzed, are more efficient, facilitate automation, and allow for the analysis of proteins that are not detectable with 2-D PAGE.

For example, in one illustrative embodiment of the present invention, the protein profile of human erythroleukemia (HEL) cells has been analyzed using the methods of the present invention as well as traditional gel based methods for comparison purposes. Two-dimensional images were generated representing each of the separation methods used. Proteins were separated and then collected using both the IEF-NP RP HPLC of the present invention and 2-D PAGE methods. These proteins were then enzymatically digested and the peptide mass maps were determined by MALDI-TOF MS (if a protein cannot be unambiguously identified by this method, further analysis is made by any number of techniques including, but not limited to, LC/MS-MS, PSD-MALDI, NMR, Western blotting, Edman sequence analysis and mass spectrometry can help with further analysis of proteins [See e.g., Yates, J. Mass Spec., 33:1 (1998); Chen et al., Rap. Comm. Mass Spec., 13:1907 (1999); Neubauer and Mann, Anal. Chem. 71:235 (1999); Zugaro et al., Electrophoresis 19:867 (1998); Immler et al., Electrophoresis 19:1015 (1998); Reid et al., Electrophoresis 19:946 (1998); Rosenfeld, et al., Anal. Biochem., 203:173 (1992); Matsui et al., Electrophoresis 18:409 (1997); Patterson and Aebersold, Electrophoresis 16:1791 (1995)]).

In some embodiments, the proteins were tentatively identified using MS-Fit to search the peptide mass maps against the Swiss and NCBInr protein databases. This work demonstrated that a large number of proteins, with a useful mass range, were separated using the methods of the present invention and that a 2-D image of these proteins was reproducibly generated for the purpose of observing distinctive patterns that are associated with a particular cell line. The methods of the present invention allowed for the detection of proteins not observed with the 2-D PAGE technique. Automation and speed of analysis are also greatly facilitated given that the proteins remain in the liquid phase throughout the separation.

In some embodiments, the present invention provides an automated protein separation and characterization system. The system is fully integrated and transfers and coordinates multi-phase, orthogonal separation methods. In some embodiments, the information is transferred by the automated system to software for the generation of multi-dimensional protein maps. Automation provides increased speed, efficiency, and sample recovery while eliminating potential sources of contamination and sample loss.

Thus, the methods of the present invention are shown to be an advantageous technique for the generation of images of protein expression profiles as well as for the collection of individual proteins for further analyses. These capabilities allow one to monitor changes in protein expression that are linked to differentiation pathways as well as particular conditions such as cancer (See e.g., Hanash, Advances in Electrophoresis; Chrambach, A., Editor, pp 1-44 [1998]), cell aging (See e.g., Steller, Science 267:1445 [1995]), the response of cells to environmental insult (See e.g., Welsh et al., Biol. Reprod., 55:141 [1996]), or the response of cells to some pharmaceutical agent. Having identified significant changes in protein expression, one can then further analyze proteins of interest to determine their identity and whether they have been altered from their expected structure by sequence changes or post-translational modifications.

Definitions

To facilitate an understanding of the present invention, a number of terms and phrases are defined below:

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the term “protein profile maps” refers to representations of the protein content of a sample. For example, “protein profile map” includes 2-dimensional displays of total protein expressed in a given cell. In some embodiments, protein profile maps may also display subsets of total protein in a cell. Protein profile maps may be used for comparing “protein expression patterns” (e.g., the amount and identity of proteins expressed in a sample) between two or more samples. Such comparing find use, for example, in identifying proteins that are present in one sample (e.g., a cancer cell) and not in another (e.g., normal tissue), or are over- or under-expressed in one sample compared to the other.

As used herein, the term “2-dimensional protein map” refers to a “protein profile map” that represents (e.g., on two axis of a graph) two properties of the protein content of a sample (e.g., including but not limited to, hydrophobicity and isoelectric point).

As used herein the term “differential display map” and equivalents “differential display plot” and “differential display image” refer to a “protein profile map” that shows the subtraction of one protein profile map from another protein profile map. A differential display map thus shows the differences in proteins present between two samples. A differential display image may also show differences in the abundance of a protein between the two samples. In some embodiments, multiple colors or color gradients are used to represent proteins from each of the two samples. An illustrative example of a differential display map is provided in Example 9 and FIG. 23.

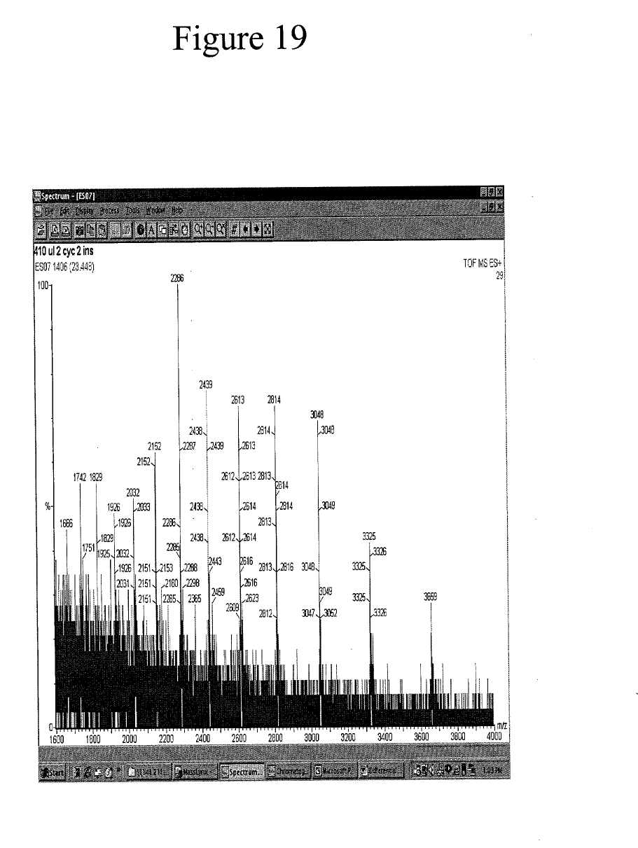

As used herein the term “deconvoluting” as in “deconvoluting mass spectrum chromatograms” refers to the processing of raw data from a mass spectrometer into “deconvoluted mass spectrum” that describe (e.g., to a computer or a human) physical parameters of proteins analyzed by the mass spectrometer (e.g., including but not limited to, protein mass and abundance). In some embodiments, “summing mass spectrum” is performed as part of “deconvoluting mass spectrum.” Example of mass spectra before and after deconvolution are shown in FIGS. 19, 20, and 21.

As used herein, the term “summing mass spectrum” refers to the process of summing a plurality of peaks on a mass spectrum. For example, summing peaks that represent multiple charge states of the same protein into one peak representing the molecular weight of the protein. As used herein, the term “summed mass spectrum” refers to mass spectrum that have been summed.

As used herein, the term “separating apparatus capable of separating proteins based on a physical property” refers to compositions or systems capable of separating proteins (e.g., at least one protein) from one another based on differences in a physical property between proteins present in a sample containing two or more protein species. For example, a variety of protein separation columns and composition are contemplated including, but not limited to ion exclusion, ion exchange, normal/reversed phase partition, size exclusion, ligand exchange, liquid/gel phase isoelectric focusing, and adsorption chromatography. These and other apparatuses are capable of separating proteins from one another based on their size, charge, hydrophobicity, and ligand binding affinity, among other properties. A “liquid phase” separating apparatus is a separating apparatus that utilizes protein samples contained in liquid solution, wherein proteins remain solubilized in liquid phase during separation and wherein the product (e.g., fractions) collected from the apparatus are in the liquid phase. This is in contrast to gel electrophoresis apparatuses, wherein the proteins enter into a gel phase during separation. Liquid phase proteins are much more amenable to recovery/extraction of proteins as compared to gel phase. In some embodiments, liquid phase proteins samples may be used in multi-step (e.g., multiple separation and characterization steps) processes without the need to alter the sample prior to treatment in each subsequent step (e.g., without the need for recovery/extraction and resolubilization of proteins).

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X,Y locations where the X and Y coordinates are defined by the first and second physical properties, respectively, or vice versa. Such displays also include multi-dimensional displays (e.g., three dimensional displays) that include additional physical properties. In some embodiments, displays are generated by “display software.”

As used herein, “characterizing protein samples under conditions such that first and second physical properties are analyzed” refers to the characterization of two or more proteins, wherein two different physical properties are assigned to each analyzed (e.g., displayed, computed, etc.) protein and wherein a result of the characterization is the categorization (i.e., grouping and/or distinguishing) of the proteins based on these two different physical properties. For example, in some embodiments, two proteins are separated based on isoelectric point and hydrophobicity.

As used herein, the term “comparing first and second physical properties of separated protein samples” refers to the comparison of two or more protein samples (or individual proteins) based on two different physical properties of the proteins within each protein sample. Such comparing includes grouping of proteins in the samples based on the two physical properties and comparing certain groups based on just one of the two physical properties (i.e., the grouping incorporates a comparison of the other physical property).

As used herein, the term “delivery apparatus capable of receiving a separated protein from a separating apparatus” refers to any apparatus (e.g., microtube, trough, chamber, etc.) that receives one or more fractions or protein samples from a protein separating apparatus and delivers them to another apparatus (e.g., another protein separation apparatus, a reaction chamber, a mass spectrometry apparatus, etc.).

As used herein, the term “detection system capable of detecting proteins” refers to any detection apparatus, assay, or system that detects proteins derived from a protein separating apparatus (e.g., proteins in one or fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, luminescence etc.) of the protein into a signal that can be processed or stored electronically or through similar means (e.g., detected through the use of a photomultiplier tube or similar system).

As used herein, the term “buffer compatible with an apparatus” and “buffer compatible with mass spectrometry” refer to buffers that are suitable for use in such apparatuses (e.g., protein separation apparatuses) and techniques. A buffer is suitable where the reaction that occurs in the presence of the buffer produces a result consistent with the intended purpose of the apparatus or method. For example, a buffer compatible with a protein separation apparatus solubilizes the protein and allows proteins to be separated and collected from the apparatus. A buffer compatible with mass spectrometry is a buffer that solubilizes the protein or protein fragment and allows for the detection of ions following mass spectrometry. A suitable buffer does not substantially interfere with the apparatus or method so as to prevent its intended purpose and result (i.e., some interference may be allowed).

As used herein, the term “automated sample handling device” refers to any device capable of transporting a sample (e.g., a separated or un-separated protein sample) between components (e.g., separating apparatus) of an automated method or system (e.g., an automated protein characterization system). An automated sample handling device may comprise physical means for transporting sample (e.g., multiple lines of tubing connected to a multi-channel valve). In some embodiments, an automated sample handling device is connected to a centralized control network.

As used herein, the term “switchable multi channel valve” refers to a valve that directs the flow of liquid through an automated sample handling device. The valve preferably has a plurality of channels (e.g., 2 or more, and preferably 4 or more, and more preferably, 6 or more). In addition, in some embodiments, flow to individual channels is “switched” on an off. In some embodiments, valve switching is controlled by a centralized control system. A switchable multi-channel valve allows multiple apparatus to be connected to one automated sample handler. For example, sample can first be directed through one apparatus of a system (e.g., a first chromatography apparatus). The sample can then be directed through a different channel of the valve to a second apparatus (e.g., a second chromatography apparatus).

As used herein, the terms “centralized control system” or “centralized control network” refer to information and equipment management systems (e.g., a computer processor and computer memory) operable linked to multiple devices or apparatus (e.g., automated sample handling devices and separating apparatus). In preferred embodiments, the centralized control network is configured to control the operations or the apparatus an device linked to the network. For example, in some embodiments, the centralized control network controls the operation of multiple chromatography apparatus, the transfer of sample between the apparatus, and the analysis and presentation of data.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “display screen” refers to a screen (e.g., a computer monitor) for the visual display of computer generated images. Images are generally displayed by the display screen as a pluarlity of pixels.

As used herein, the term “computer system” refers to a system comprising a computer processor, computer memory, and a display screen in operable combination. Computer systems may also include computer software.

As used herein, the term “directly feeding” a protein sample from one apparatus to another apparatus refers to the passage of proteins from the first apparatus to the second apparatus without any intervening processing steps. For example, a protein that is directly fed from a protein separating apparatus to a mass spectrometry apparatus does not undergo any intervening digestion steps (i.e., the protein received by the mass spectrometry apparatus is undigested protein).

As used herein, the term “sample” is used in its broadest sense. In one sense it can refer to a cell lysate. In another sense, it is meant to include a specimen or culture obtained from any source, including biological and environmental samples. Biological samples may be obtained from animals (including humans) and encompass fluids, solids, tissues, and gases. Biological samples include blood products (e.g., plasma and serum), saliva, urine, and the like and includes substances from plants and microorganisms. Environmental samples include environmental material such as surface matter, soil, water, and industrial samples. These examples are not to be construed as limiting the sample types applicable to the present invention.

### DETAILED DESCRIPTION OF THE INVENTION

The present invention provides a novel multi-dimensional separation method that is capable of resolving large numbers of cellular proteins. The following discussion is provided in four sections: I) two-phase separation techniques; II) improved elution techniques; III) mass spectroscopic analysis and 2-D display systems and methods; and IV) automated 3D HPLC/MC methods for rapid protein characterization.

I) Two-Phase Separation Techniques

The first dimension separates proteins based on a first physical property. For example, in some embodiments of the present invention proteins are separated by pI using isoelectric focusing in the first dimension (See e.g., Righetti, Laboratory Techniques in Biochemistry and Molecular Biology; Work, T. S.; Burdon, R. H., Elsevier: Amsterdam, p 10 [1983]). However, the first dimension may employ any number of separation techniques including, but not limited to, ion exclusion, ion exchange, normal/reversed phase partition, size exclusion, ligand exchange, liquid/gel phase isoelectric focusing, and adsorption chromatography. In some embodiments (e.g., some automated embodiments), it is preferred that the first dimension be conducted in the liquid phase to enable products of the separation step to be fed directly into a second liquid phase separation step.

The second dimension separates proteins based on a second physical property (i.e., a different property than the first physical property) and is preferably conducted in the liquid phase (e.g., liquid-phase size exclusion). For example, in some embodiments of the present invention proteins are separated by hydrophobicity using non-porous reversed phase HPLC in the second dimension (See e.g., Liang et al., Rap. Comm. Mass Spec., 10:1219 [1996]; Griffin et al., Rap. Comm. Mass Spec., 9:1546 [1995]; Opiteck et al., Anal. Biochem. 258:344 [1998]; Nilsson et al., Rap. Comm. Mass Spec., 11:610 [1997]; Chen et al., Rap. Comm. Mass Spec., 12:1994 [1998]; Wall et al., Anal. Chem., 71:3894 [1999]; Chong et al., Rap. Comm. Mass Spec., 13:1808 [1999]). This method provides for exceptionally fast and reproducible high-resolution separations of proteins according to their hydrophobicity and molecular weight. The non-porous (NP) silica packing material used in these reverse phase (RP) separations eliminates problems associated with porosity and low recovery of larger proteins, as well as reducing analysis times by as much as one third. Separation efficiency remains high due to the small diameter of the spherical particles, as does the loadability of the NP RP HPLC columns. However, the second dimension may employ any number of separation techniques. For example, in one embodiment, 1-D SDS PAGE lane gel is used. Having the second dimension conducted in the liquid phase facilitates efficient analysis of the separated proteins and enables products to be fed directly into additional analysis steps (e.g., directly into mass spectrometry analysis).

In certain embodiments of the present invention, proteins obtained from the second separation step are mapped using software (available from Dr. Stephen J. Parus, University of Michigan, Department of Chemistry, 930 N. University Ave., Ann Arbor, Mich. 48109-1055) in order to create a protein pattern analogous to that of the 2-D PAGE image—although based on the two physical properties used in the two separation steps rather than by a second gel-based size separation technique. In some embodiments, RP HPLC peaks are represented by bands of different intensity in the 2-D image, according to the intensity of the peaks eluting from the HPLC. In some embodiments, peaks are collected as the eluent of the HPLC separation in the liquid phase.

In some embodiments, the proteins collected from the second dimension were identified using proteolytic enzymes, MALDI-TOF MS and MSFit database searching. In an example using human erythroleukemia cell lysate, using IEF-NP RP HPLC, approximately 700 bands were resolved in a pI range from 3.2 to 9.5 and 38 different proteins with molecular weights ranging from 12 kDa to 75 kDa were identified. In comparison to a 2-D gel separation of the same human erythroleukemia (HEL) cell line lysate, the IEF-NP RP HPLC produced improved resolution of low mass and basic proteins. In addition, the proteins remained in the liquid phase throughout the separation, thus making the entire procedure highly amenable to automation and high throughput.

Certain preferred embodiments are described in detail below. These illustrative examples are not intended to limit the scope of the invention. For example, although the examples are described using human tissues and samples, the methods and apparatuses of the present invention can be used with any desired protein samples including samples from plants and microorganisms.

A. IEF-NP RP HPLC Method

The following description provides certain preferred embodiments for conducting isoelectric separation (first dimension) and NP RP HPLC separation (second dimension) according to the methods of the present invention.

1. IEF Separation

Proteins are extracted from cells using a lysis buffer. To facilitate an efficient process, this lysis buffer should be compatible with the downstream separation and analysis steps (e.g., NP RP HPLC and MALDI-TOF-MS) to allow direct use of the products from each step into subsequent steps. Such a buffer is an important aspect of automating the process. Thus, the preferred buffer should meet two criteria: 1) it solubilizes proteins and 2) it is compatible with each of the steps in the separation/analysis methods. Although the present invention provides suitable buffers for use in the particular method configurations described below, one skilled in the art can determine the suitability of a buffer for any particular configuration by solubilizing protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

After extraction in the lysis buffer, proteins are initially separated in a first dimension. The goal in this step is that the proteins are isolated in a liquid fraction that is compatible with subsequent NP RP HPLC and mass spectrometry steps. In these embodiments, n-octyl β-D-glucopyranoside (OG1, from Sigma) is used in the buffer. n-octyl β-D-glucopyranoside is one of the few detergents that is compatible with both NP RP HPLC and subsequent mass spectrometry analyses. It is contemplated that detergents of the formula n-octyl SUGARpyranoside find use in these embodiments. The lysis buffer utilized was 6M urea, 2M thiourea, 1.0% n-octyl β-D-glucopyranoside, 10 mM dithioerythritol and 2.5% (w/v) carrier ampholytes (3.5 to 10 pI)). After extraction, the supernatant protein solution is loaded to a device that can separate the proteins according to their pI by isoelectric focusing (IEF). Here the proteins are solubilized in a running buffer that again should be compatible with NP RP HPLC. A suitable running buffer is 6M urea, 2M thiourea, 0.5% n-octyl β-D-glucopyranoside, 10 mM dithioerythritol and 2.5% (w/v) carrier ampholytes (3.5 to 10 pI).

Three exemplary devices that may be used for this step are:

a) Rotofor

This device (Biorad) separates proteins in the liquid phase according to their pI (See e.g., Ayala et al., Appl. Biochem. Biotech. 69:11 [1998]). This device allows for high protein loading and rapid separations that require only four to six hours to perform. Proteins are harvested into liquid fractions after a 5-hour IEF separation. These liquid fractions are ready for analysis by NP RP HPLC. This device can be loaded with up to 1 g of protein.

b) Carrier Ampholyte Based Slab Gel IEF Separation with a Whole Gel Eluter

In this case the protein solution is loaded onto a slab gel and the proteins separate in to a series of gel-wide bands containing proteins of the same pI. These proteins are then harvested using a whole gel eluter (WGE, from Biorad). Proteins are then isolated in liquid fractions that are ready for analysis by NP RP HPLC. This type of gel can be loaded with up to 20 mg of protein.

c) IPG Slab Gel IEF Separation with a Whole Gel Eluter

Here the proteins are loaded onto a immobiline pI gradient slab gel and separated into a series of gel-wide bands containing proteins of the same pI. These proteins are electro-eluted using the WGE into liquid fractions that are ready for analysis by NP RP HPLC. The IPG gel can be loaded with at least 60 mg of protein.

2. Protein Separation by NP RP HPLC

Having obtained liquid fractions containing large amounts of pI-focused proteins, the second dimension separation is non-porous RP HPLC. The present invention provides the novel combination of employing non-porous RP packing materials (Eichrom) with another RP HPLC compatible detergent (e.g., n-octyl β-D-galactopyranoside) to facilitate the multi-phase separation of the present invention. This detergent is also compatible with mass spectrometry due to its low molecular weight. The use of these types of RP HPLC columns for protein separations as a second dimension separation after IEF in order to obtain a 2-D protein separation is a novel feature of the present invention. These columns are well suited to this task as the non-porous packing they contain provides optimal protein recovery and rapid efficient separations. It should be noted that though several detergents have been mentioned thus far for increasing protein solubility while being compatible with RP HPLC there are many other different low molecular weight non-ionic detergents that could be used for this purpose. Several important features that allow the RP HPLC to work as a second dimension are as follows: The mobile phase should contain a low level of a non-ionic low molecular weight detergent such as n-octyl β-D-glucopyranoside or n-octyl β-D-galactopyranoside as these detergents are compatible with RP HPLC and also with later mass spectrometry analyses (unlike many other detergents); the column should be held at a high temperature (around 60° C.); and the column should be packed with non-porous silica beads to eliminate problems of protein recovery associated with porous packings.

3. Protein Detection and Identification via Mass Spectrometry

In some embodiments of the present invention, the products of the second separation step are further characterized using mass spectrometry. For example, the proteins that elute from the NP RP HPLC separation are analyzed by mass spectrometry to determine their molecular weight and identity. For this purpose the proteins eluting from the separation can be analyzed simultaneously to determine molecular weight and identity. A fraction of the effluent is used to determine molecular weight by either MALDI-TOF-MS or ESI oa TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI oa TOF. The molecular weight 2-D protein map is matched to the appropriate digest fingerprint by correlating the molecular weight total ion chromatograms (TIC's) with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

4. Automation

All of the above described steps are automated, for example, into one discrete instrument. In one illustrative embodiment, the first dimension is carried out by a Rotofor, with the harvested liquid fractions being directly applied to the second dimension non-porous RP HPLC apparatus through the appropriate tubing. The products from the second dimension separation are then scanned and the data interpreted and displayed as a 2-D representation using the appropriate computer hardware and software. Alternately, the products from the second dimension fractions are sent through the appropriate microtubing to a mass spectrometry pre-reaction chamber where the samples are treated with the appropriate enzymes to prepare them for mass spectrometry analysis. The samples are then analyzed by mass spectrometry and the resulting data is received and interpreted by a processor. The output data represents any number of desired analyses including, but not limited to, identity of the proteins, mass of the proteins, mass of peptides from protein digests, dimensional displays of the proteins based on any of the detected physical criteria (e.g., size, charge, hydrophobicity, etc.), and the like. In preferred embodiments, the proteins samples are solubilized in a buffer that is compatible with each of the separation and analysis units of the apparatus. Using the automated systems of the present invention provides a protein analysis system that is an order of magnitude less expensive than analogous automation technology for use with 2-D gels (See e.g., Figeys and Aebersold, J. Biomech. Eng. 121:7 [1999]; Yates, J. Mass Spectrom., 33:1 [1998]; and Pinto et al., Electrophoresis 21:181 [2000]).

5. Software and Data Presentation

The data generated by the above listed techniques may be presented as 2-D images much like the traditional 2-D gel image. In some embodiments, the chromatograms, TIC's or integrated and deconvoluted mass spectra are converted to ASCII format and then plotted vertically, using a 256 step gray scale, such that peaks are represented as darkened bands against a white background. The scale could also be in a color format. The image generated by this method provides information regarding the pI, hydrophobicity, molecular weight and relative abundance of the proteins separated. Thus the image represents a protein pattern that can be used to locate interesting changes in cellular protein profiles in terms of pI, hydrophobicity, molecular weight and relative abundance. Naturally the image can be adjusted to show a more detailed zoom of a particular region or the more abundant protein signals can be allowed to saturate thereby showing a clearer image of the less abundant proteins. This information can be used to assess the impact of disease state, pharmaceutical treatment, and environmental conditions. As the image is automatically digitized it may be readily stored and used to analyze the protein profile of the cells in question. Protein bands on the image can be hyper-linked to other experimental results, obtained via analysis of that band, such as peptide mass fingerprints and MSFit search results. Thus all information obtained about a given 2-D image, including detailed mass spectra, data analyses, and complementary experiments (e.g., immuno-affinity and peptide sequencing) can be accessed from the original image.

The data generated by the above-listed techniques may also be presented as a simple read-out. For example, when two or more samples are compared (See, Section J, below), the data presented may detail the difference or similarities between the samples (e.g., listing only the proteins that differ in identity or abundance between the samples). In this regard, when the differences between samples (e.g., a control sample and an experimental sample) are indicative of a given condition (e.g., cancer cell, toxin exposure, etc.), the read-out may simply indicate the presence or identity of the condition. In one embodiment, the read-out is a simple +/−indication of the presence of particular proteins or expression patterns associated with a specific condition that is to be analyzed.

6. IEF-NP RP HPLC in Operation

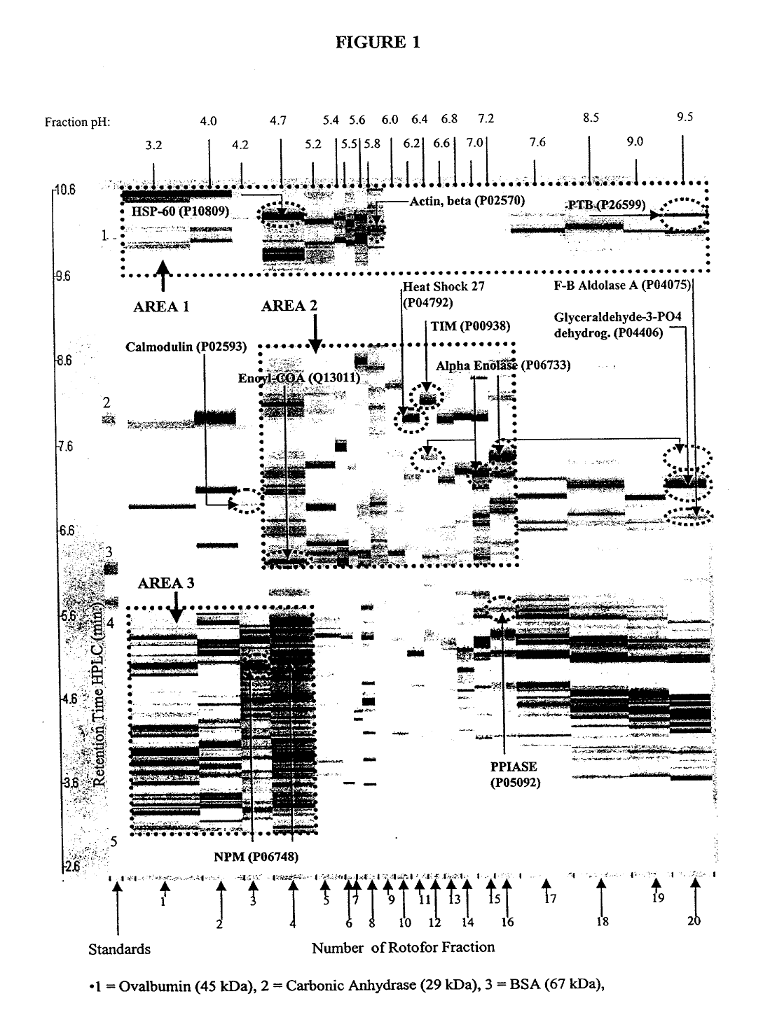

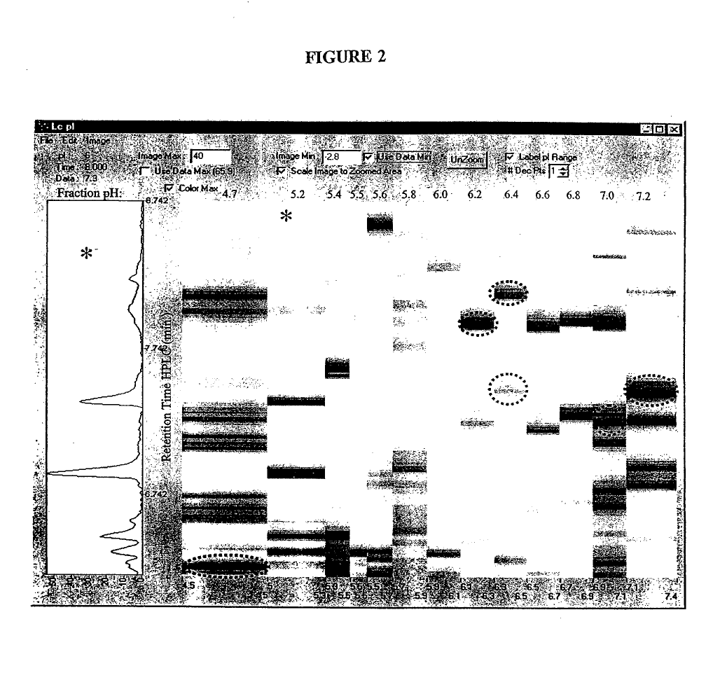

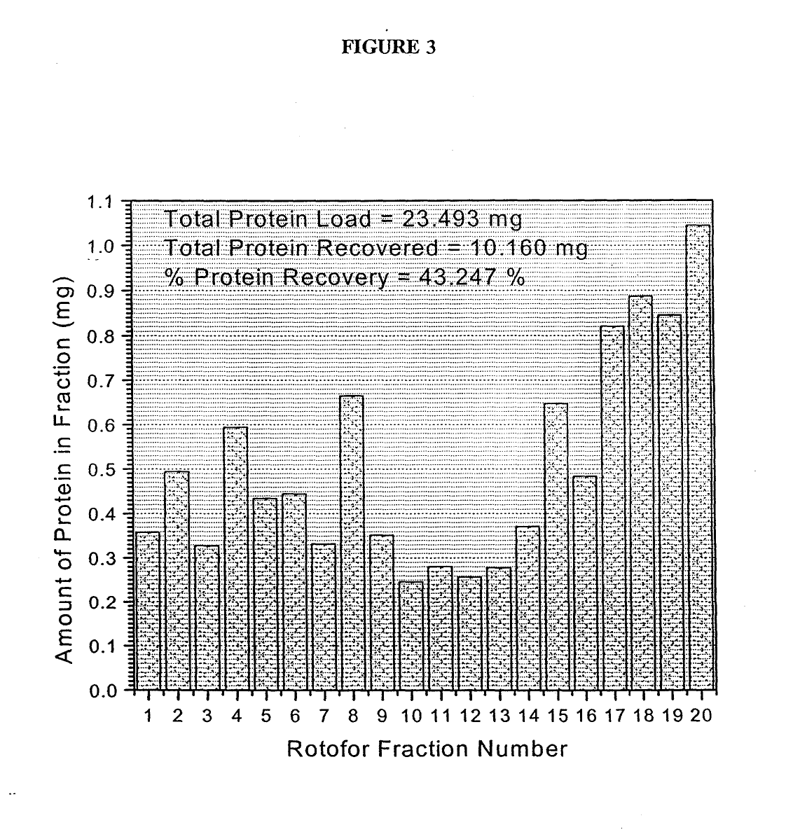

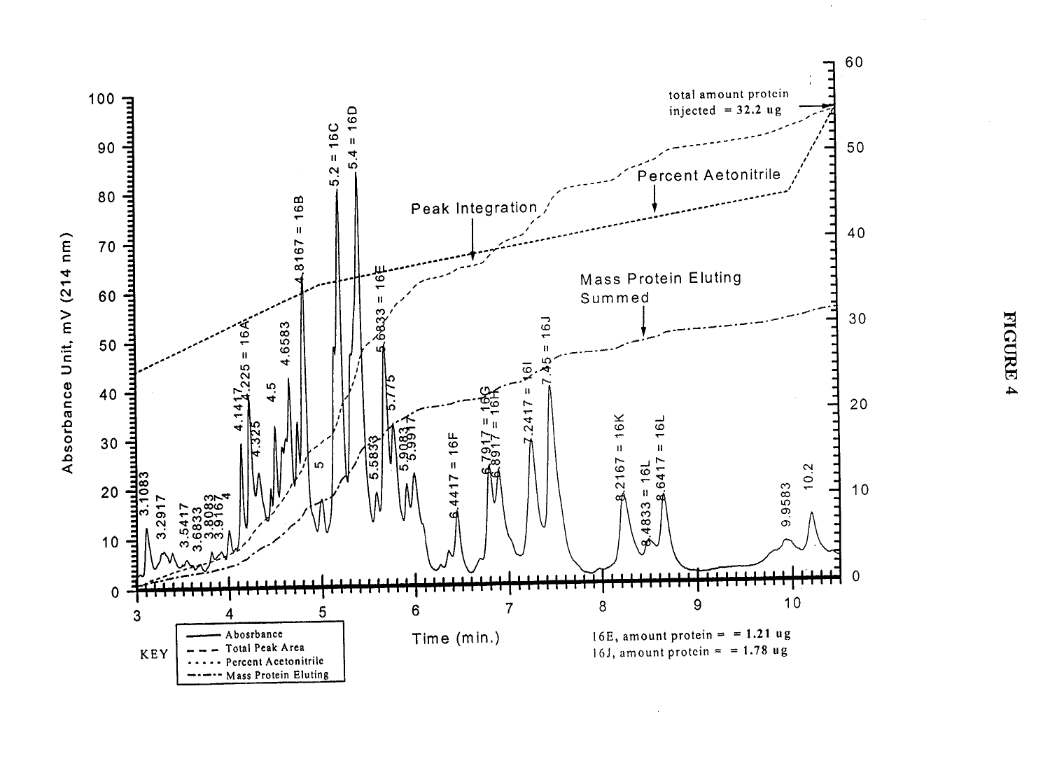

The IEF-NP RP HPLC image shown in FIG. 1 is a digital representation of a 2-dimensional separation of a whole cell protein lysate from a human erythroleukemia (HEL) cell line. This image is designed to offer the same advantages of pattern recognition and protein profiling that may be obtained using a 2-D gel. The horizontal and vertical dimensions are in terms of isoelectric point and protein hydrophobicity, respectively. The isoelectric focusing step, performed using the Rotofor, resulted in 20 protein fractions ranging in pH from 3.2 to 9.5. These fractions were then injected onto a non-porous reversed phase column for separation by HPLC and detection by UV absorbance (214 nm). The resulting chromatograms were converted to ASCII format and then plotted vertically, using a 256 step gray scale, such that peaks are represented as darkened bands against a white background. Protein profiles may be viewed in greater detail by using the zoom feature as shown in FIG. 2 and/or by selecting a particular Rotofor fraction and observing the NP RP HPLC chromatogram as shown in the left panel of FIG. 2. The zoom and chromatogram image features provide a means to observe details in band patterns that may not be observable in the original image (See, FIG. 1). In addition, because of the limitations of the 256 step gray scale representation the band intensities in areas 1, 2 and 3 of FIG. 1 were rescaled by a factor of 3 to better show the low abundance proteins. This was preferred since the presence of several high abundance protein bands may cause low intensity bands in some regions to be undetected. In FIG. 1, the total peak area for each individual chromatogram was scaled to reflect the relative amount of protein that was found in the original Rotofor fraction (See, FIG. 3). The band intensities in different chromatograms can therefore be compared directly thus providing a true image of relative protein abundance in the cell lysate. The width of the Rotofor fraction columns was adjusted to represent their estimated pH range. The molecular weight of proteins observed by IEF-NP RP HPLC ranged from 12 kDa to 75 kDa. Typical NP RP HPLC separations, as shown in FIG. 4, resulted in 35 peaks in 10.5 minutes. The total number of peaks that could be observed from all 20 fractions is estimated to be approximately 700.

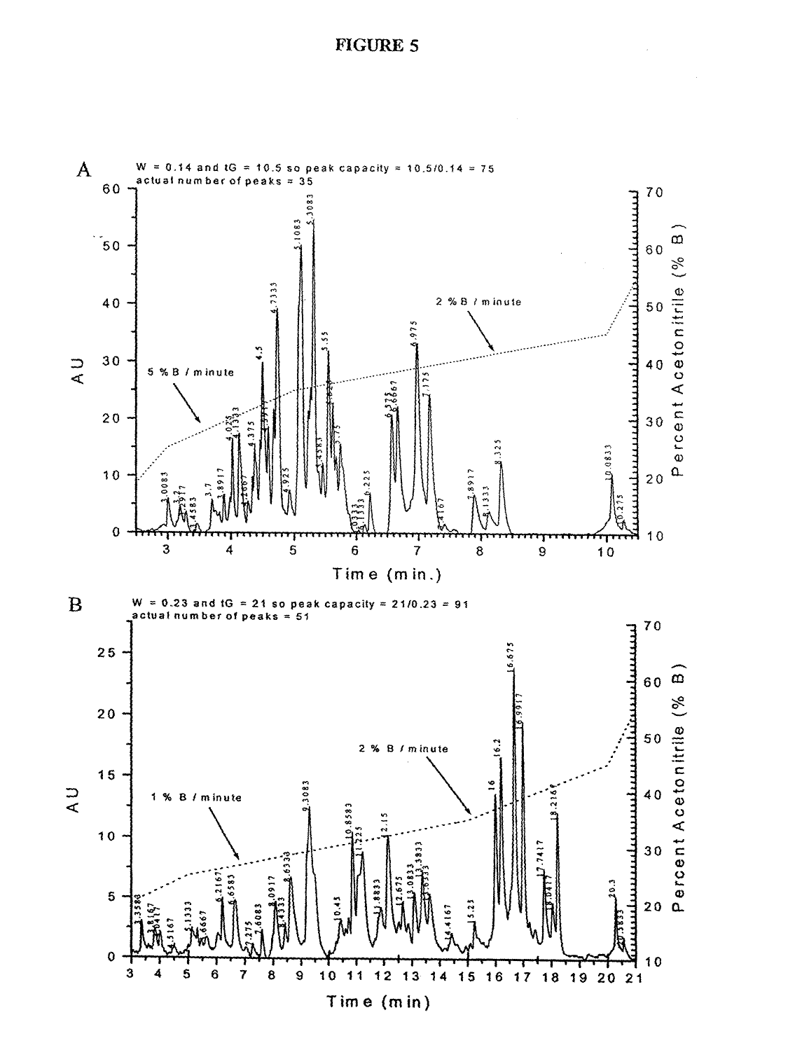

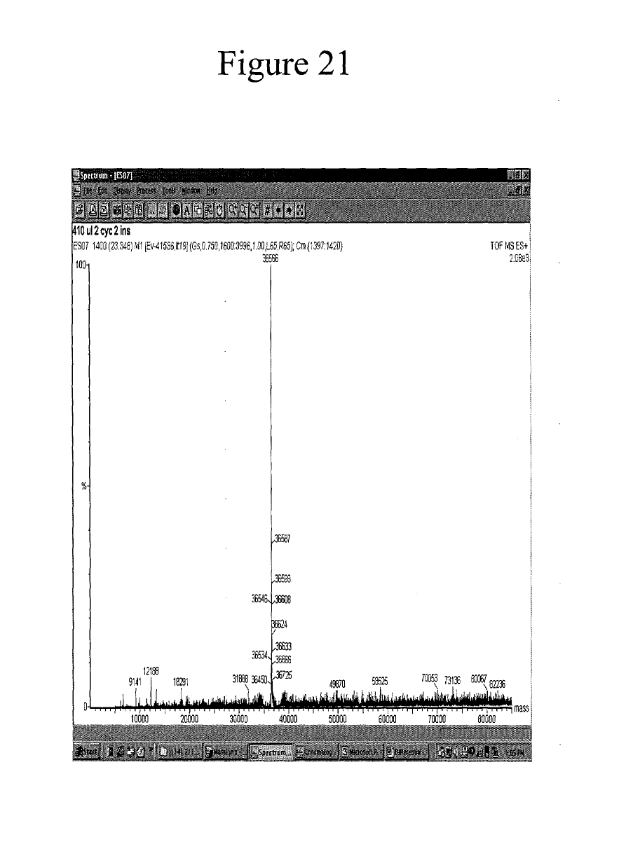

The gradient time (t_{G}) used in the above experiments is very short and a significant increase in peak capacity is expected with longer gradients. This is shown using Rotofor fraction 17 where two separations were performed with gradient times of 10.5 minutes (See, FIG. 5A) and 21 minutes (See, FIG. 5B). With t_{G}=10.5 minutes, the average peak width was 0.14 minutes and the peak capacity was therefore 75. The actual number of peaks resolved was 35. With t_{G}=21 minutes the average peak width was 0.23 minutes and the peak capacity was therefore 91. The actual number of peaks resolved was 51. Using the longer separation time with t_{G}=21 minutes the total number of peaks observed should increase from 700 to 1000. However, it should be noted that when using mass spectrometric detection, that sufficient resolution should be available to ultimately resolve the same number of peaks without using a longer gradient time.

The proteins in a representative sampling of these peaks were identified using the traditional approach of enzymatic digestion, MALDI-TOF MS peptide mass analysis and MSFit database searching. The magnification of the IEF-NP RP HPLC image enables the viewer to perceive more bands than is possible to observe from the whole image. In addition, as shown in FIG. 2, the viewer may select a particular band format chromatogram and observe the traditional peak format of the chromatogram in a window to the left of the image. This allows the observer to use the peak format chromatogram to find partially resolved peaks that may not be observable in the band format chromatogram. Five standard protein bands are shown in the left-most column where the masses range from 14.2 kDa up to 67 kDa. As RP HPLC separates proteins by hydrophobicity, these standards are not molecular weight markers as in a traditional 1-D gel. Rather, they are used to indicate the range of protein molecular weights that may be observed. Ten different proteins are labeled on the image although many more proteins were identified as shown in Table 1, below. In some embodiments of the present invention, where it is desired that certain proteins or classes of proteins are to be detected, the starting protein sample may be selectively labeled. After the proteins are passed through the separation step, detection of the proteins can be limited to those that contain the selective label.

B. Protein Separation by 2-D SDS PAGE

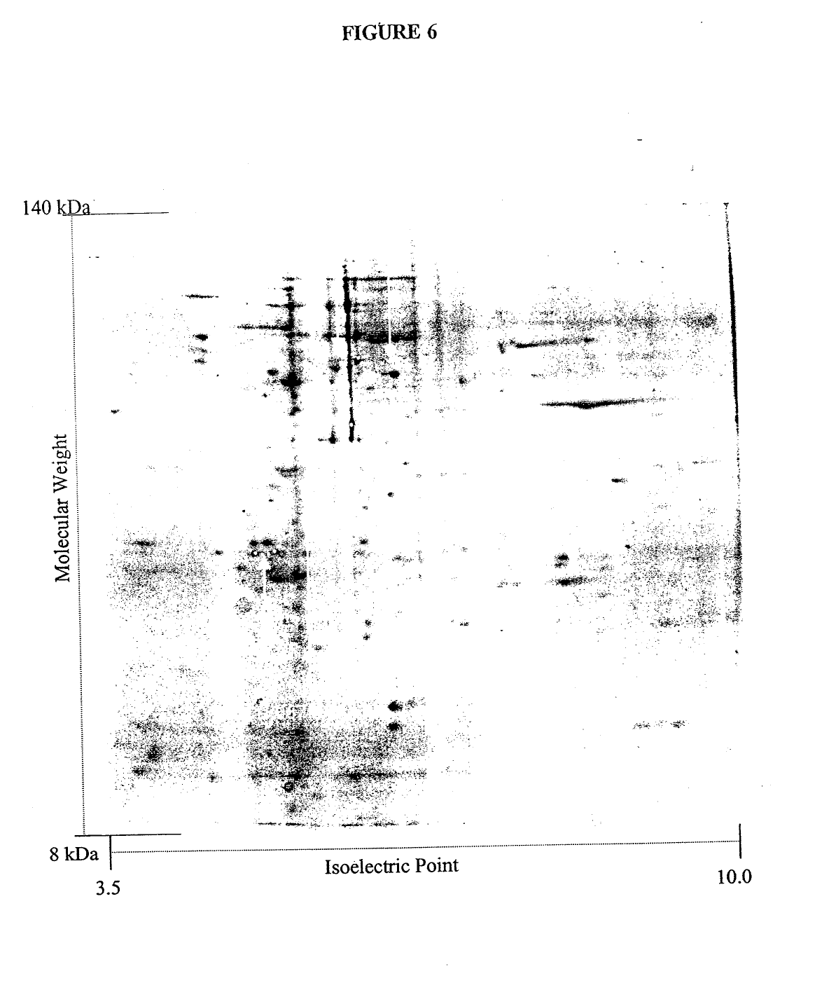

The image in FIG. 1 represents the IEF-NP RP HPLC separation of the HEL cell protein lysate and the image in FIG. 6 represents the Coomassie blue (CBB) stained 2-D SDS PAGE separation of the same HEL cell line lysate. The pI range for this gel is the same as that used for the Rotofor separation and the molecular weight range is from 8 kDa to 140 kDa. As with the IEF-NP RP HPLC separation a representative sampling of the isolated proteins was identified using enzymatic digestion, MALDI-TOF MS and MSFit methods (See e.g., Rosenfeld et al., Anal. Biochem. 203:173 [1992]). For the target protein mass range of this study (10 kDa-70 kDa) approximately 188 protein spots are observed on the CBB stained gel, 355 from the CBB stained polyvinylidene difluoride (PVDF) blot, and 652 from the silver stained gel as estimated using BioImage 2D Analyzer Version 6.1 software (Genomic Solutions). The total spot capacity for the 2-D gel separation is estimated to be 2100. The proteins identified from the gel are labeled on the image and also shown in Table 2, below. An image of another 2-D gel separation of HEL cell proteins can be observed via the Swiss-2DPAGE database (See e.g., http://www.expasy.ch; Sanchez et al, Electrophoresis 16:1131 [1995]). In addition, it is possible to view the latest protein list for the HEL cell in which 19 protein entries are shown (See e.g., http://www.expasy.ch/cgi-bin/get-ch2d-table.pl).

TABLE 1
Thirty Eight Proteins Identified From HEL Cell IEF-NP RP HPLC Separation

| Rotofor - | | Retention | | MWt/pI: database | Swiss, NCBInr | |
|---|---|---|---|---|---|---|
| Fraction # | pH | Time (min.) | Enzyme* | calculated | Accession # | Protein Name |
| 3 | 4.20 | 5.34 | trypsin | 32575.2/4.64 | P06748 | NPM |
| 3 | 4.20 | 6.20 | trypsin | 11665.0/4.42 | P05387 | 60S RIBOSOMAL PROTEIN P2 |
| 3 | 4.20 | 6.91 | trypsin | 16637.7/4.09 | P02593 | CALMODULIN |
| 3 | 4.20 | 10.15 | trypsin | 41737.0/5.29 | P02570 | BETA-ACTIN & GAMMA ACTIN |
| 3 | 4.20 | 10.25 | trypsin | 61055.0/5.70 | P10809 | HSP-60 |
| 4 | 4.70 | 5.38 | trypsin | 32575.2/4.64 | P06748 | NPM |
| 4 | 4.70 | 6.24 | trypsin | 35994.6/6.61 | Q13011 | ENOYL-COA HYDRATASE |
| 4 | 4.70 | 7.07 | trypsin | 57914.2/7.95 | P14786 | PYRUVATE KINASE, M2 |
| 4 | 4.70 | 10.28 | trypsin | 61055.0/5.70 | P10809 | HSP-60 |
| 5 | 5.40 | 4.93 | trypsin | 22988.1/5.10 | P52566 | RHO GDI 2 |
| 5 | 5.40 | 10.15 | trypsin | 70898.4/5.38 | P11142 | HEAT SHOCK COGNATE 71 KD PROTEIN |
| 8 | 5.60 | 4.99 | trypsin | 22988.1/5.10 | P52566 | RHO GDP-DISSOCIATION INHIBITOR 2 |
| 8 | 5.60 | 7.94 | trypsin | 69224.5/5.49 | P23588 | EIF-4B |
| 8 | 5.60 | 0.35 | trypsin | 49831.3/4.79 | P05217 | TUBULIN BETA-2 CHAIN |
| 9 | 5.80 | 6.90 | trypsin | 56782.7/5.99 | P30101 | ERP60 |
| 9 | 5.80 | 8.05 | trypsin | 17148.8/5.83 | P15531 | METASTASIS INHIBITION FACTOR NM23 |
| 9 | 5.80 | 8.50 | trypsin | 26669.6/6.45 | P00938 | TRIOSEPHOSPHATE ISOMERASE (TIM) |
| 9 | 5.80 | 10.15 | trypsin | 41737.0/5.29 | P02570 | BETA-ACTIN & GAMMA ACTIN |
| 11 | 6.20 | 5.62 | trypsin | 36926.7/6.37 | 5542020 | (L32610) ribonucleoprotein |
| 11 | 6.20 | 7.65 | trypsin | 33777.2/6.26 | 4885153 | (X59656) CRKL |
| 11 | 6.20 | 7.91 | trypsin | 22327.3/7.83 | P04792 | HEAT SHOCK 27 |
| 11 | 6.20 | 8.80 | trypsin | 74674.0/8.51 | Q92935 | EXOSTOSIN-L |
| 11 | 6.20 | 9.22 | trypsin | 37374.9/5.85 | P19883 | FOLLISTATIN 1 AND 2 PRECURSOR |
| 11 | 6.20 | 10.40 | trypsin | 47033.1/5.30 | 5032183 | cargo selection protein TIP47 |
| 12 | 6.40 | 5.08 | trypsin | 13802.0/6.43 | P49773 | HINT |
| 12 | 6.40 | 5.90 | trypsin | 70021.3/5.56 | P54652 | HEAT SHOCK 70 KD PROTEIN 2 |
| 12 | 6.40 | 7.48 | trypsin | 47169.2/7.01 | P06733 | ALPHA ENOLASE |
| 12 | 6.40 | 8.12 | trypsin | 26669.6/6.45 | P00938 | TRIOSEPHOSPHATE ISOMERASE (TIM) |
| 13 | 6.60 | 4.88 | trypsin | 48058.0/5.34 | P05783 | KERATIN, TYPE I CYTOSKELETAL 18 |
| 13 | 6.60 | 8.28 | trypsin | 62639.6/6.40 | P31948 | TRANSFORMATION-SENSITIVE PROTEIN |
| 13 | 6.60 | 8.65 | trypsin | 34902.4/7.42 | 4505059 | carcinoma-associated antigen GA733-2 |
| 15 | 7.00 | 4.70 | trypsin | 37429.9/3.97 | P22626 | NUCLEAR RIBONUCLEOPROTEINS A2/B1 |
| 15 | 7.00 | 8.70 | trypsin | 22391.6/8.41 | P37802 | SM22-ALPHA HOMOLOG |
| 15 | 7.00 | 7.25 | trypsin | 47169.2/7.01 | P06733 | ALPHA ENOLASE |
| 16 | 7.20 | 5.68 | trypsin, Glu-C (E) | 18012.6/7.68 | P05092 | PPIASE |
| 16 | 7.20 | 6.89 | trypsin | 35940.7/7.18 | P01861 | IG GAMMA-4 CHAIN C REGION |
| 16 | 7.20 | 7.14 | trypsin | 36053.4/8.57 | P04406 | GLYCERALDEHYDE 3-PHOSPHATE |
| 16 | 7.20 | 7.45 | trypsin, Glu-C (E) | 47169.2/7.01 | P06733 | ALPHA ENOLASE |
| 16 | 7.20 | 8.64 | trypsin, Glu-C (E) | 22391.6/8.41 | P37802 | SM22-ALPHA HOMOLOG |
| 19 | 9.00 | 4.88 | trypsin | 33846.0/9.26 | P09651 | NUCLEAR RIBONUCLEOPROTEIN A1 |
| 19 | 9.00 | 5.13 | trypsin | 37429.9/8.97 | P22626 | NUCLEAR RIBONUCLEOPROTEINS A2/B1 |
| 19 | 9.00 | 5.85 | trypsin | 46987.1/7.58 | P13929 | BETA ENOLASE |
| 19 | 9.00 | 7.47 | trypsin | 36053.4/8.57 | P04406 | GLYCERALDEHYDE 3-PHOSPHATE |
| 19 | 9.00 | 8.70 | trypsin | 38604.2/7.58 | P07355 | ANNEXIN II |
| 19 | 9.00 | 9.07 | trypsin | 22391.6/8.41 | P37802 | SM22-ALPHA HOMOLOG |
| 19 | 9.00 | 10.53 | trypsin | 57221.6/9.22 | P26599 | PTB, NUCLEAR RIBONUCLEOPROTEIN I |
| 20 | 9.50 | 4.46 | trypsin, Glu-C (E) | 38846.0/9.26 | P09651 | NUCLEAR RIBONUCLEOPROTEIN A1 |
| 20 | 9.50 | 4.67 | trypsin, Glu-C (E) | 37429.9/8.97 | P22626 | NUCLEAR RIBONUCLEOPROTEINS A2/B1 |
| 20 | 9.50 | 6.72 | trypsin, Glu.C (E) | 39420.2/8.30 | P04075 | FRUCTOSE-BISPHOSPHATE ALDOLASE A |
| 20 | 9.50 | 7.06 | trypsin | 36053.4/8.57 | P04406 | GLYCERALDEHYDE 3-PHOSPHATE |
| 20 | 9.50 | 7.39 | trypsin, Glu-C (E) | 47169.2/7.01 | P06733 | ALPHA ENOLASE |
| 20 | 9.50 | 8.52 | trypsin, Glu-C (E) | 22391.6/8.41 | P37802 | SM22-ALPHA HOMOLOG |
| 20 | 9.50 | 10.16 | trypsin | 44728.1/8.30 | P00558 | PHOSPHOGLYCERATE KINASE I |
| 20 | 9.50 | 10.35 | trypsin | 57221.6/9.22 | P26599 | PTB, NUCLEAR RIBONUCLEOPROTEIN I |

*Note that all proteins labelled only with trypsin were not digested with Glu-C (E)

TABLE 2
Nine Proteins Identified From HEL Cell CBB 2-D Gel

| Gel Spot I.D. | | MWt/pI: database | SwissProt | |
|---|---|---|---|---|
| Number | Enzyme | calculated | Accession # | Protein Name |
| g1 | trypsin | 18012.6/7.68 | P05092 | PPIASE |
| g2 | trypsin | 26669.6/6.45 | P00938 | TRIOSEPHOSPHATE ISOMERASE (TIM) |
| g3 | trypsin | 26669.6/6.45 | P00938 | TRIOSEPHOSPHATE ISOMERASE (TIM) |
| g8 | trypsin | 29032.8/4.75 | P12324 | TROPOMYOSIN, CYTOSKELETAL TYPE (TM30-NM) |
| g10 | trypsin | 32575.2/4.64 | P06748 | NPM |
| g11 | trypsin | 41737.0/5.29 | P02570 | BETA-ACTIN |
| g12 | trypsin | 61055.0/5.70 | P10809 | HSP-60 |
| g13 | trypsin | 56782.7/5.99 | P30101 | ERP60 |
| g14 | trypsin | 47169.2/7.01 | P06733 | ALPHA ENOLASE |

C. IEF-NP RP HPLC versus 2-D SDS PAGE: Protein Loading and Quantification

Each separation method relies upon orthogonal mechanisms of separation generating a large number of isolated proteins. Protein profiles may be compared in terms of their pattern as well as the relative amounts of isolated proteins. It is shown, however, that the loadability of the liquid phase methods of the present invention greatly surpasses that of the gel phase.

The limit of detection for the gel method when stained with the silver stain is approximately 1 to 10 ng. The Coomassie blue stain can detect 100 ng of protein and the amount of protein in the spot can be quantified over 2.5 orders of magnitude. For the NP RP HPLC of standard proteins used in certain embodiments of the methods of the present invention, the limit of detection for the UV detector was 10 ng. The protein in the peak can be quantified from 10 ng up to 20 μg providing 3.1 orders of magnitude. Quantification of an HPLC peak involves integrating the peak to find the area. For the gel, the spots must first be digitized and then this image must be analyzed to determine the integrated optical density of each spot of interest. The sensitivity of the UV detector in embodiments of the present invention utilizing HPLC is competitive with the silver stain and quantification is much simpler. The limits of detection for both the silver stained gel and the HPLC UV peak detection are mass dependent. For the gel, resolution and sensitivity are proportional to the molecular weight of the protein. For IEF-NP RP HPLC, the resolution and sensitivity are inversely proportional to the molecular weight of the protein. The gel appears to provide improved results for both acidic proteins and proteins above 50 kDa whereas IEF-NP RP HPLC performs better with proteins in the basic region and proteins that are below 50 kDa (See e.g., FIG. 1 and FIG. 6). These results show the complementary nature of these two techniques where the gel and IEF-NP RP HPLC each provide important information of protein content.

In one experiment using the methods of the present invention, 23.5 mg of protein was loaded into the Rotofor, and after a five-hour IEF separation period fractions ranging from 2 to 4 mL were collected into polypropylene microtubes. The amount of protein in the individual fractions ranged from 0.25 mg to 1.05 mg. Summing the amounts of protein in each fraction led to the determination that a total of 10.2 mg of protein was recovered from the Rotofor. This amount can be increased by increasing the amount of non-ionic detergent in the Rotofor buffer above the current 0.1% level as well as by the addition of thiourea. In contrast, the amount of protein loaded on the 2-D gel in FIG. 6 is 200 μg. The amount of protein that actually makes it through the gel and focuses to a spot has not been quantified, relative to the amount of protein that is actually loaded on the gel, though it is known that many hydrophobic proteins are lost during the separation (Herbert, Electrophoresis 20:660 [1999]). The amount of protein that may theoretically be loaded on a gel ranges from 5 μg up to 250 μg whereas for IEF-NP RP HPLC the initial loading of protein may be as high as 1 gram. The amount of protein actually used to produce the separation shown in FIG. 1 is only a fraction of the amount initially loaded into the Rotofor. The image in FIG. 1 actually represents the separation of a total of 1 to 2 mg of protein though 10.2 mg of protein was recovered from the Rotofor. The loading of the HPLC column being used currently could be increased though the peak capacity may suffer. Alternatively a larger column could be used in series with the smaller column to allow for higher loadability with no loss of separation efficiency (See e.g., Wall et al., Anal. Chem., 71:3894 [1999]).

A 2-D gel provides a two dimensional separation from one initial loading of the cell lysate. The intensities of different spots on the same gel are representative of the relative protein abundances in the original lysate. However, in the IEF-NP RP HPLC methods of the present invention the proteins are loaded for the IEF and the HPLC separations so that the band intensities in the 2-D IEF-NP RP HPLC image depend on the amount of protein loaded to the HPLC from each Rotofor fraction. Since the amount of material in each Rotofor fraction is different, the total area of each chromatogram was scaled to represent the total amount of protein that was recovered for each Rotofor fraction (See, FIG. 3). The result is that the protein band intensities can be compared both within the Rotofor fraction and between the different fractions.

In some embodiments of the present invention, 2-D gel techniques are used side-by-side with IEF-NP RP HPLC. In embodiments where specific proteins are desired for further characterization, the gel can provide information indicating which fraction obtained with IEF-NP RP HPLC contains the desired protein or proteins.

D. Isoelectric Focusing: Liquid vs. Gel Phase

The principal concern with liquid phase IEF is that the protein is not isoelectrically focused as effectively as it would be in a gel due to diffusion of the protein in solution. In the case of α-enolase, if one compares the liquid and gel phase images, it can be seen that in both cases substantial spreading of the protein occurs over a wide pI range. This range spans from pI 6.5 to pI 9.5 in both the liquid phase and the gel phase. For more acidic proteins such as β-actin, it appears that in the liquid phase the protein is more dispersed in the pI dimension than for the corresponding gel separated protein. Both methods provide a reasonably accurate assessment of the pI of the protein of interest. Referring to Table 1, it can be seen that as the Rotofor fraction pH increases, so generally does the pI of identified proteins therein. The pH of fraction 3 measures 4.2 and the proteins identified from this fraction range in pI from 4.09 to 5.7. The pH of fraction 9 was 5.8 and the proteins identified from that fraction ranged from 5.29 to 6.45. The pH of fraction 16 was 7.2 and the pI range of proteins found there ranged from 7.01 to 8.93. The pI accuracy therefore ranges from +/−0.65 to 1.73 pI units. This is comparable to the carrier ampholyte based gel. It should be remembered that the pI of a given protein may vary significantly due to post-translational modifications such as phosphorylation and glycosylation, as well as to artifactual modifications such as carbamylation and oxidation.

E. Second Dimension Liquid Separation

Fraction 16, FIG. 4, may be used as an example of the quantification of isolated proteins. For fraction 16, the volume of injection was 160 μL. This means that if the concentration of protein was 201.4 μg/mL then the amount of protein loaded was 32.2 μg. The chromatogram was integrated using Microcal Origin software and the total area was determined to be 97.78. The areas of peaks 16E and 16J were 3.68 and 5.41 respectively. Dividing the peak area by the total area gives the fraction of protein represented by the peak. Therefore, if one assumes 100% protein recovery, the amount of PPIASE (16E, t_{R}=5.68) in 16 was (0.0376*32.2 μg) 1.21 μg and the amount of α-enolase (16J, t_{R}=7.45) was (0.0553*32.3 μg) 1.78 μg. The peak areas were generated by absorbance of 214 nm light at the amide bonds of the proteins and so should offer low selectivity thereby allowing for a good measure of the amount of protein in the peak regardless of the type of protein.

FIG. 4 shows how the continuous integration of the chromatogram may be used to estimate the amount of protein isolated in a given peak. The peak area line is simply converted into mass units from which the observer can measure the change in the vertical mass axis that occurs over the width of the peak of interest. If one knows the initial concentration of protein in the cell lysate and the number of cells that were lysed, a quantitative comparison of different cell lysates can be made. This comparison is important to studying changes in protein expression levels due to some disease state or pharmacological treatment. In gel work, a technique used for protein quantification in different samples is to normalize the integrated optical density of the spot of interest to that of standard proteins whose expression levels are thought to be constant. In this way any experimental variation in spot intensity can be corrected. This same method is applied to the IEF-NP RP HPLC image to allow for reliable quantification of proteins of interest such that changes in expression level are quantitatively observed.

The assumption in these experiments is 100% protein recovery. One can determine the actual % recovery of protein and the dependence on elution time. Typical protein recoveries have been shown to range from 70 to 95% in NP RP HPLC (Wall et al., Anal. Chem., 71:3894 [1999]) and so, with a more likely percent recovery of 80%, the amount of PPIASE and α-enolase in fraction 16 would be estimated to be 1.0 μg and 1.42 μg, respectively.

F. Rotofor Fraction Analysis by NP RP HPLC vs. 1-D SDS PAGE

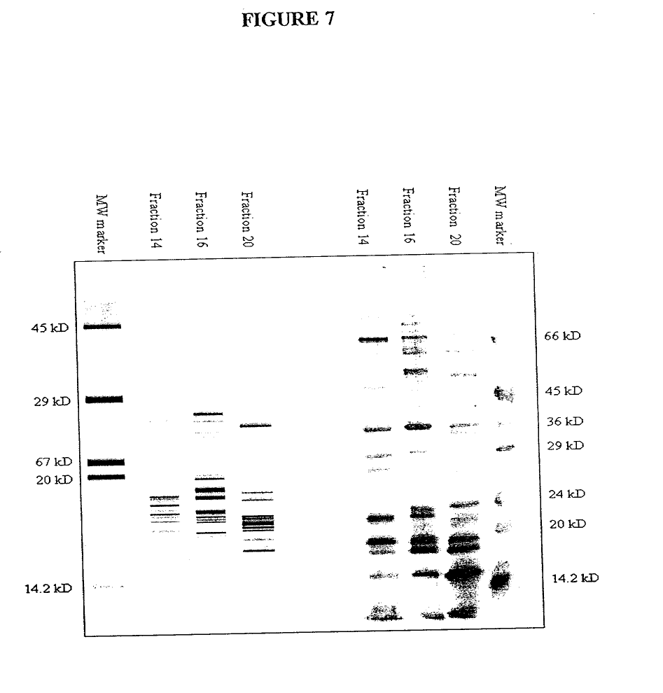

NP RP HPLC provides highly efficient protein separations (See e.g., Chen et al., Rap. Comm. Mass Spec., 12:1994 [1998]; Wall et al., Anal. Chem., 71:3894 [1999]; and Chong et al., Rap. Comm. Mass Spec., 13:1808 [1999]), and is a far easier method to automate as compared to gels in terms of injection, data processing and protein collection. In addition the NP RP HPLC separations provided by the present invention are 70 times faster than the equivalent separation by 1-D SDS-PAGE, which requires 14 hours. In the experiments described above, the NP RP HPLC method has greater resolving power generating 35 bands where the 1-D gel generates only 26 bands. A direct comparison of the two methods, as shown in FIG. 7, reveals that the NP RP HPLC bands are much narrower than those of the 1-D SDS PAGE over a similar molecular weight range. Also it is clear that as molecular weight decreases, the 1-D gel band width increases substantially. In NP RP HPLC the opposite trend occurs where the lower molecular weight proteins show improved resolution and sensitivity. This image may appear to show that the NP RP HPLC separation fails with larger proteins as there are few bands in the upper region of the image. However, this is not the case as it is important to remember that the vertical dimension for NP RP HPLC is not protein molecular weight but rather protein hydrophobicity. This is evidenced by the observation of the elution of bovine serum albumin (66 kDa), a relatively hydrophilic protein, half way up an image.

G. Elution Time Prediction for Known Target Protein

One of the advantages of the 2-D gel is that the vertical coordinate of the gel may be used to estimate the molecular weight of the protein with a +/−10% error. The position of a protein of interest can therefore be estimated before the protein is identified from the gel. In an attempt to correlate elution time in the methods of the present invention with the mass of the protein, a linear fit to a plot of percent acetonitrile at time of elution (% B) versus the log(MWt)/protein polar ratio was generated. The polar ratio (PR) is the number of polar amino acids divided by the total number of amino acids in the protein and the molecular weight is in kDa. The proteins used for this plot were four of the standards listed in FIG. 1 as well as a sampling of six of the proteins from Table 1 (HSP60, β-actin, TIM, α-enolase, PPIASE and glyceraldehyde-3-phosphate). The resulting equation (equation 1: % B/100=0.079805*(logMWt)/PR+0.077686, (R=0.9677, SD=0.014722, N=7)) is used to predict the elution time of target proteins. For HSP60, β-actin and α-enolase the experimental elution times were 10.28, 10.15 and 7.25 respectively. The predicted elution times were 10.20, 10.13 and 9.78. In the cases of HSP60 and β-actin the prediction works well, whereas for α-enolase the prediction is not as good. While not precise, this prediction does give some idea of when a protein will elute such that a given target protein, for which the molecular weight and hydrophobicity are known, can be found more readily.

H. Protein Identification by Enzymatic Digestion, MALDI-TOF MS and MSFit Database Searching

The proteins that were identified from a representative sampling of the bands from the IEF-NP RP HPLC separation are listed in Table 1. A sampling of approximately 80 proteins from 12 of the Rotofor fractions were digested and their peptide mass maps successfully obtained by MALDI-TOF MS. Of these 80, 38 different proteins were identified. In this case, identifying roughly 50% of the proteins searched is to be expected as not all the proteins are in the available databases. Similar results were observed for proteins analyzed from 2-D gels of the HEL cell samples. The current table in Swiss-2DPAGE lists 19 protein entries for the HEL cell. Of these 19 proteins, five were identified from the IEF-NP RP HPLC separation. In the gel, these same five proteins were also identified.

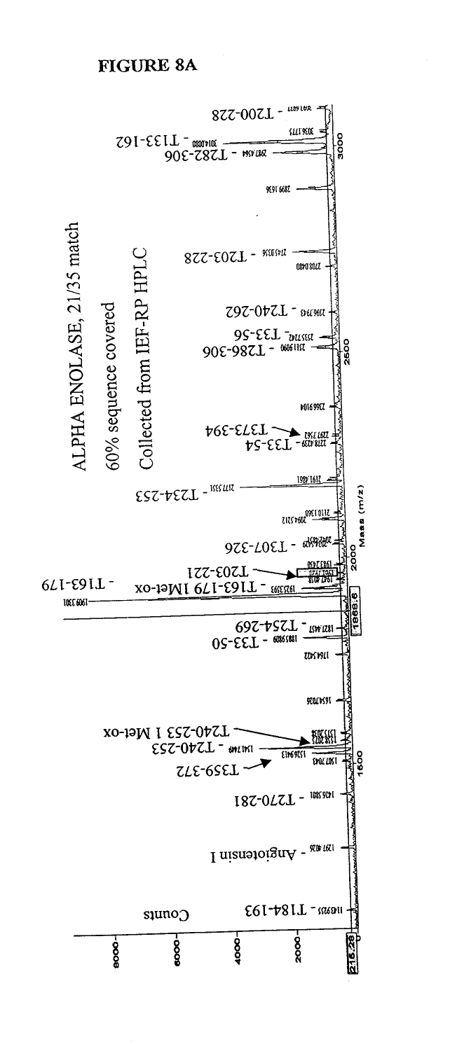

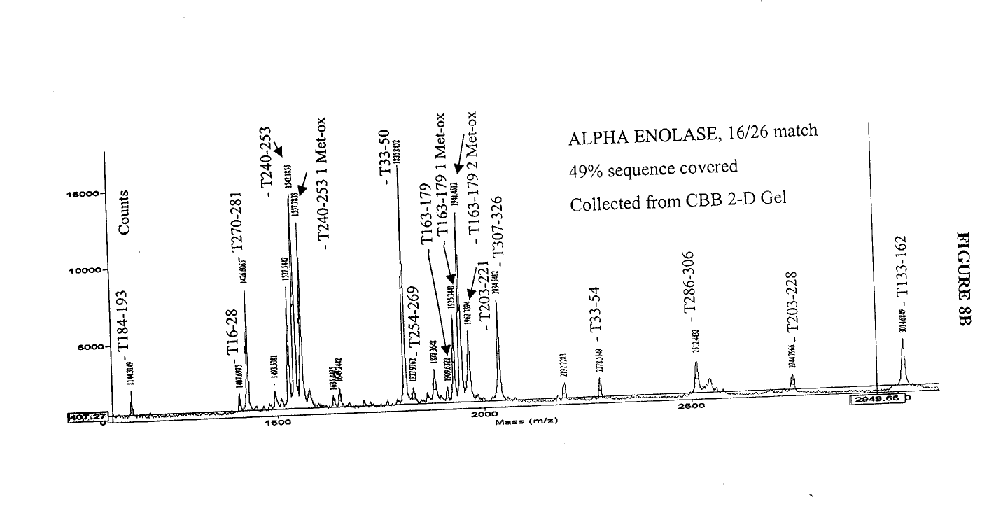

In general, it appears that the gel MSFit results are better than those from the liquid phase. This can be attributed to the fact that the gel proteins were reduced and alkylated with DTE and iodoacetamide respectively prior to the running of the second dimension. This step would help insure that all disulfide bonds are broken and optimal proteolysis is produced. Thus, this derivatization step can be added to the IEF-NP RP HPLC method, by performing the reduction and alkylation step prior to NP RP HPLC or during cell lysis. Nevertheless, in some cases the IEF-NP RP HPLC digestions surpassed those from the gel in coverage and quality. This is evidenced in FIG. 8, which shows a direct comparison of the MALDI-TOF MS for α-enolase as isolated via the IEF-NP RP HPLC method and the gel method. These mass spectra were calibrated externally at first and the mass profiles used to search the Swiss protein database with a mass accuracy of 400 ppm. These searches gave strong hits to α-enolase for both the gel and the liquid protein digests. Each mass spectrum was then recalibrated internally using matched peptide peaks from the initial externally calibrated match. The new peak table was then used to search the same Swiss protein database but with 200 ppm mass accuracy. FIG. 8 clearly shows that the digestion from the liquid phase is improved compared to that from the gel. The IEF-NP RP HPLC mass spectrum matches to 60% of the protein sequence whereas that from the gel matches to 49%. Achieving a match to 60% of the sequence of a 47 kDa protein is very unusual for MALDI-TOF MS analysis and represents a significant improvement over gel digests. Although the present invention is not limited to any particular mechanism, the increase in sequence coverage may be due to the fact that the protein is digested in the liquid phase, is relatively pure, and because the peptides are not lost due to being embedded inside the gel piece. Also if one observes the level of methionine oxidation in the peak that matches to T163-179, it is clear that the protein isolated by IEF-NP RP HPLC is far less oxidized than that from the gel.

Many of the NP RP HPLC chromatograms contain some peaks that are not fully resolved to baseline. This need not be a problem as partially resolved proteins can still be effectively identified using MALDI-TOF MS analysis. In Rotofor fraction 3 there are peaks at 10.15 minutes and 10.25 minutes (See, Table 1). These peaks are only resolved to 50% above the baseline and yet it is clear that the peak eluting at 10.15 minutes is β-actin and the peak eluting at 10.25 minutes is HSP-60. Note that the predicted elution times for these proteins are 10.13 and 10.20 minutes respectively. As proteins can be identified from partially resolved peaks, faster separations with more rapid gradients are possible. The reproducibility of the pattern of bands can be determined by looking at the retention times for particular proteins as observed from different Rotofor fractions. β-actin elutes at 10.15 minutes in both fractions 3 and 9; α-enolase elutes at 7.25, 7.45 and 7.39 minutes in fractions 12, 16 and 20 respectively; and HSP-60 elutes at 10.28 and 10.25 minutes in fractions 3 and 4 respectively. Clearly, with +/−0.1 minutes variation in the retention times, these separations are quite reproducible from run to run.

Thus, the methods of the present invention have been shown to provide advantageous methods for the reproducible separation of large numbers of proteins. In the human erythroleukemia cell lysate example, the methods are capable of resolving 700 bands with a rapid gradient, and 1000 bands with a longer gradient. There were 38 different proteins tentatively identified, by MALDI-TOF MS and MSFit database searching, after analysis of a fraction of these bands. This compares favorably with the 19 different proteins that have been identified to date from the 2-D gel. Some of the proteins found in the human erythroleukemia cell lysate; including oc-enolase (Rasmussen et al., Electrophoresis 19:818 [1998] and Mohammad et al., Enz. Prot., 48:37 [1994]), glyceraldehyde-3-phosphate dehydrogenase (Bini et al., Electrophoresis 18:2832 [1997] and Sirover, Biochim. Biophys. Acta 1432:159 [1999]), NPM (Redner et al., Blood 87:882 [1996]), CRKL (ten Hoeve et al., Oncogene 8:2469 [1993]), and heat shock protein (HS27) (Fuqua et al., Cancer Research 49:4126 [1989]), have been linked to various forms of cancer. NPM and CRKL have been linked specifically to leukemias.

The proteins identified in one exemplary experiment ranged from 12 kDa up to 75 kDa (although broader ranges are contemplated by the present invention); this range may include many of the proteins of interest to current research involving protein profiling, identification and correlation to some disease state or cell treatment. In sharp contrast to 2-D gels, this method is well-suited to automation. Mass spectrometric methods can be applied, such as ESI-MS and MALDI-TOF MS, to the detection of whole proteins and protein digests. Most importantly, the methods of the present invention provide an alternative 2-D protein map to the traditional 2-D gel and appears to improve results for lower mass proteins and more basic proteins. A key advantage of the liquid 2-D separation is that the end product is a purified protein in the liquid phase. Also, since the initial protein load can be fifty times that of the gel, the amount of a target protein that may be isolated by one IEF-NP RP HPLC separation is potentially fifty times higher than that obtainable from a 2-D gel separation. Additionally, in the case that the investigator is interested in specific proteins where the pI is known, this method may be used to isolate and identify the target protein in less than 24 hours, since only the fraction of interest need be analyzed via the second dimension separation. The gel-based method would require three days to achieve the same result.

I. Identification of Novel Tumor Antigens

There is substantial interest in identifying tumor proteins that are immunogenic. Autoantibodies to tumor antigens and the antigens themselves represent two types of cancer markers that can be assayed in patient serum and other biological fluids. IEF-NP RP HPLC-MS has been implemented for the identification of tumor proteins that elicit a humoral response in patients with cancers. The identification of proteins that specifically react with sera from cancer patients was demonstrated using this approach. Solubilized proteins from a tumoral cell line are subjected to IEF-NP RP HPLC-MS. Individual fractions defined on the basis of pI range are subjected simultaneously to one-dimensional electrophoresis as well as to HPLC. Sera from cancer patients are reacted with Western blots of one-dimensional electrophoresis fractions. One band which reacted specifically with sera from lung cancer patients and not from controls was found to contain both Annexin II and aldoketoreductase. The ability to subfractionate further proteins contained in this fraction by HPLC led to the identification of Annexin II as the tumor antigen that elicited a humoral response in lung cancer patients.

J. Comparative Analysis

As is clear from the above description, the methods of the present invention offer the opportunity to compare protein profiles between two or more samples (e.g., cancer vs. control cells, undifferentiated vs. differentiated cells, treated vs. untreated cells). In one embodiment of the present invention, the two samples to be compared are run in parallel. The data generated from each of the samples is compared to determine differences in protein expression between the samples. The profile for any given cell type may be used as a standard for determining the identity of future unknown samples. Additionally, one or more proteins of interest in the expression pattern may be further characterized (e.g., to determine its identity). In an alternative embodiment, the proteins from the samples are run simultaneously. In these embodiments, the proteins from each sample are separately labeled so that, during the analysis stage, the protein expression patterns from each sample are distinguished and displayed. The use of selective labeling can also be used to analyze subsets of the total protein population, as desired.

As is clear from the above description, the methods and compositions of the present invention provide a range of novel features that provide improved methods for analyzing protein expression patterns. For example, the present invention provides methods that combine IEF, resulting in pI-focused proteins in liquid phase fractions, with nonporous RP HPLC to produce 2-dimensional liquid phase protein maps. The data generated from such methods may be displayed in novel and useful formats such as viewing a collection of different pI NP RP HPLC chromatograms in one 2-D image displaying the chromatograms in a top view protein band format, not the traditional side view peak format. As shown in FIG. 2, the side view peak format is shown to the left and the top view band format is shown to the right. The present invention also provides detergents that are compatible with automated systems employing multi-phase separation and detection steps.

The present invention provides additional characterization steps, including the identification of proteins separated by IEF-NP RP HPLC using enzymatic digestions and mass spectrometric analysis of the resulting peptide mass fingerprints. Proteins may be detected to determine their molecular weights by analyzing the effluent from the HPLC with either off-line collection to a MALDI plate (Perseptive) or on-line analysis using orthogonal extraction time-of-flight. The data generated from such methods may be displayed in novel and useful formats such as using the data from the MALDI or LCT generated protein molecular weights to generate total ion chromatograms (TIC) that would be virtually identical to the original UV-absorbance chromatograms. The signal of these chromatograms would be based on the number of ions generated from the HPLC effluent of a given group of pI-focused proteins, not by absorption of light. These chromatograms are plotted in the same 2-D top view band format as mentioned above. These methods allow one to fully integrate and deconvolute each of the TIC's generated to display complete mass spectra of each collection of pI-focused proteins. The methods also allow the display of all the integrated TIC's in one 2-D image where the vertical dimension is in terms of protein molecular weight and the horizontal dimension is in terms of protein pI. The protein mass spectra appears as bands as they are also viewed from the top. This image would therefore also contain quantitative information (in the case of the LCT) and so the bands would vary in intensity depending on the amount of protein present.

The liquid phase methods for protein mass mapping would also allow for collection of protein fractions to microtubes such that the proteins could be digested and the peptide mass maps analyzed to determine the identity of said proteins simultaneously. Laser induced fluorescence (LIF) detection schemes are used in conjunction with this method to increase the overall sensitivity by three orders of magnitude. The liquid phase LIF detector provides more sensitive fluorescence detection than in the gel as there would be no gel background fluorescence. This LIF detection method could be used in a number of ways including, but not limited to:

1) Combining equal amounts of two cell lysates that have each been previously stained with a different fluorescent dye followed by use of a dual fluorescence detector to simultaneously detect the same proteins from two different cell lysates. This would allow for very accurate comparisons of the relative amounts of proteins found for different cell lines or tissues; and

2) Using a fluorescently tagged antibody to label specific target proteins in a cell lysate such that they can be targeted for thorough analysis without looking at all the other proteins.

The methods and apparatuses of the present invention also offer an efficient system for combining with other analysis techniques to obtain a thorough characterization of a given cell, tissue, or the like. For example, the methods of the present invention may be used in conjunction with genetic profiling technologies (e.g., gene chip or hybridization based nucleic acid diagnostics) to provide a fuller understanding of the genes present in a sample, the expression level of the genes, and the presence of protein (e.g., active protein) associated with the sample.

II) Improved Elution Techniques Using Chromatofocusing

As described above, the present invention provides novel liquid chromatographic methods involving a 2-column 2-D separation of proteins from whole cell lysates followed by on-line mass mapping with by mass spectrometry (e.g., using ESI-oaTOF MS as described in detail below). It is a 3-D protein analysis system as proteins are separated based upon, for example, their isoelectric points (pI) in the first LC dimension.

The present invention further provides novel techniques for eluting proteins from a separation apparatus (e.g., the first phase separation apparatus). For example, in one embodiment of this technique, the proteins eluted from the first dimension are “peeled off” from the column according to their pH, either one pH unit or fraction thereof, at a time—referred to as chromatofocusing (CF). These focused liquid fractions are then separated according to their hydrophobicity and size (or other desired properties) in the second dimension. Liquid fractions from, for example, NP-RP-HPLC can be conveniently analyzed directly on-line using mass spectrometry (e.g., ESI-oaTOF) to obtain their molecular weight and relative abundance, which provides a third dimension. As a result, a virtual 2-D protein image is created and is analogous to a 2-D gel image. Furthermore, this 2-D protein image includes vital information such as the pI, hydrophobicity, molecular weight, and relative abundance. This “Protein Peeling” 2-D LC-MS method is a practical alternative to 2-D gels in order to study protein expression between normal and disease whole cell lysates, for example.

This whole system can be fully automated and integrated into a single unit for rapid proteome analysis, providing a more accurate and less expensive automation technology compared to automation technologies for use with 2-D gels.

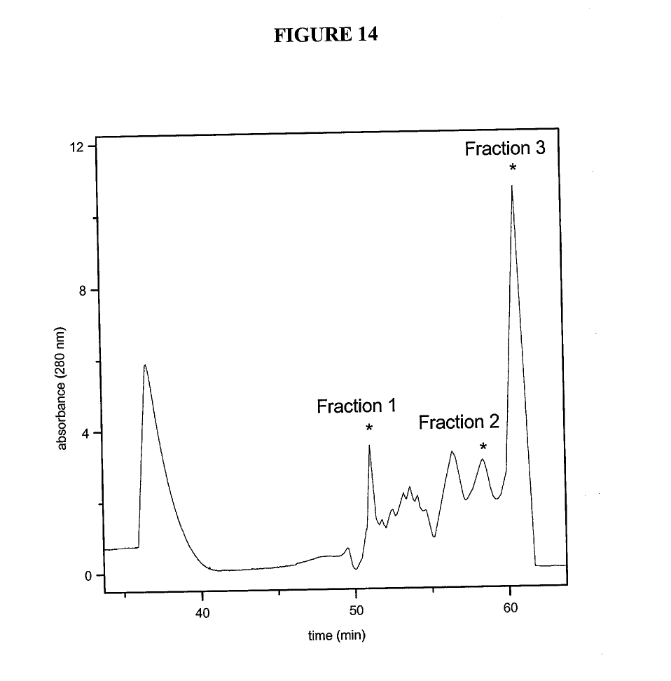

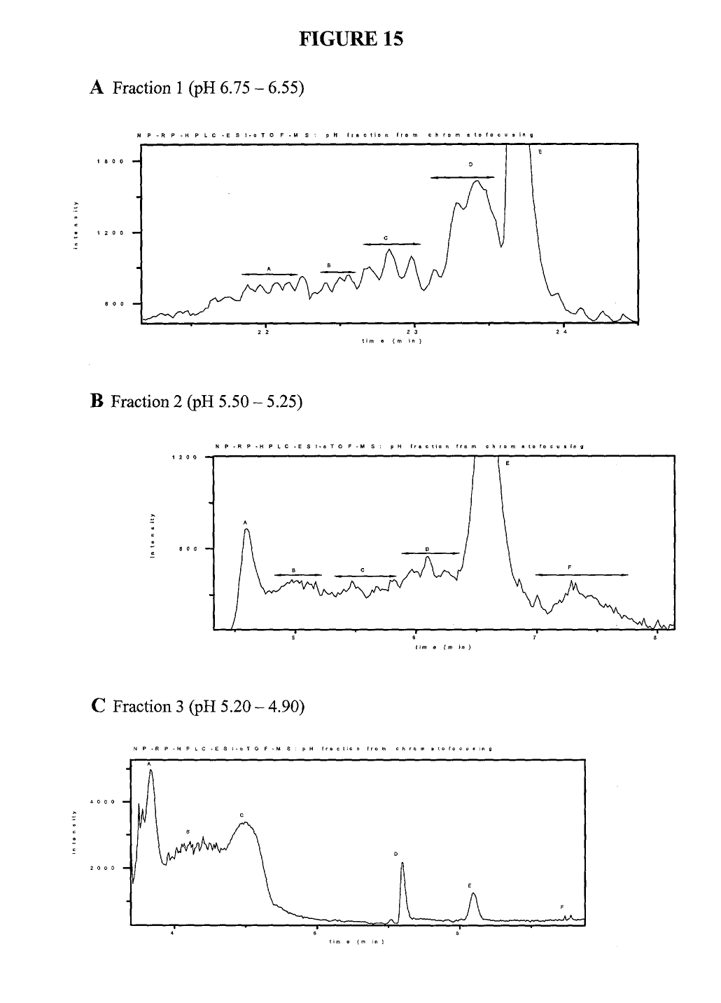

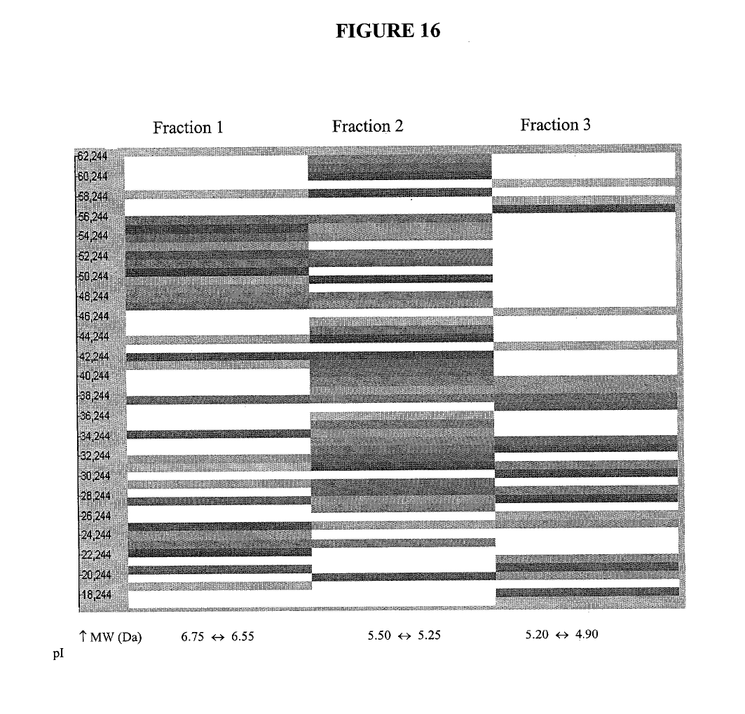

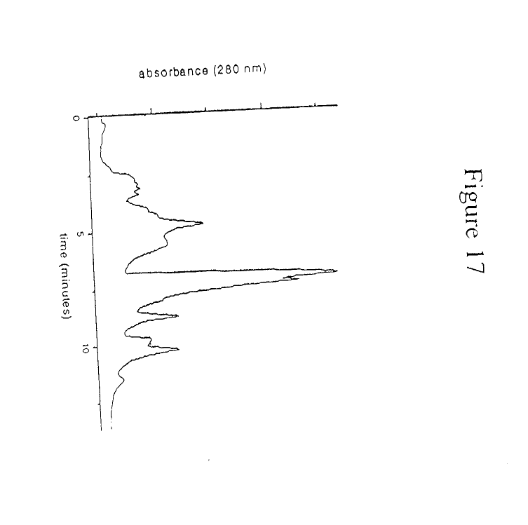

An exemplary embodiment of the chromatofocusing techniques of the present invention are provided in Example 7. Data from these experiments is shown in FIGS. 14-16. FIG. 14 shows the CF profile of MCF-10A whole cell lysate (pH 7 to 4). Fractions 1 to 3 were further analyzed with NP-RP-HPLC-ESI-oaTOF MS (described in detail below). FIGS. 15A-C show the NP-RP-HPLC-ESI-oaTOF TIC (total ion count) profile of the three fractions from FIG. 14: (A) fraction 1 (pH 6.75-6.55); (B) fraction 2 (pH 5.50-5.25); and (C) fraction 3 (pH 5.20-4.90). By integrating and deconvoluting the TIC profiles with the MaxEnt1 software (described in detail below), the mass spectra for all three fractions are displayed in a 2-D format as shown in FIG. 16. FIG. 16 shows the integrated TIC in one 2-D protein map where the vertical column is the molecular weight while the horizontal dimension is the protein pI point. This map also contains the relative abundance information whereby the bands vary in intensity (shades of gray) depending on the amount of the protein present.

The data generated by CF-NP-RP-HPLC-ESI-oaTOF MS can be presented as 2-D maps or 2-D images much like the traditional 2-D gel images. For example, in some embodiments, the chromatograms, TICs, integrated and deconvoluted mass spectra are converted into the ASCII format before being plotted vertically, using a 256-step gray scale, such that peaks are represented as darkened bands against a white background. This scale comes in a variety of color formats. Therefore, this 2-D map provides vital information on pI, hydrophobicity, molecular weight as well as the relative abundance of separated proteins. This map can also be adjusted by zoom into a specific area of interest, for a more detailed image of all the bands therein. All the information gathered from this 2-D map can be used to examine protein expression in a cell system due to the disease state, pharmaceutical treatment or environmental change. Since the image is automatically digitized, it can be easily stored and the bands can be hyperlinked to other experimental results or related data. As a result, all the information is available from the original image.

The use of chromatofocusing with the separation, analysis, and display methods of the present invention provide a number of important advantages not previously available. For example, by combining chromatofocusing with a second separation phase (e.g., NP-RP-HPLC) and mass spectrometry analysis, a 2-D liquid phase protein map is generated which is analogous to a 2-D gel. In preferred embodiments, this is a multi-dimensional liquid chromatography (LC) whereby both chromatographic techniques are performed on-line (i.e., in an automated fashion) between two or multiple LC units with a switching valve to deliver fractions from CF to, for example, NP-RP-HPLC. Proteins are “peeled off” the CF column according to their pH, one pH unit or fraction thereof, at a time. This “peeling” feature allows for further focusing of the protein bands at their respective pI regions. The protein concentration of each pI band is thus enhanced during elution. As with the method described above, buffers can be used that are compatible with each step of the process. For example, in some embodiments, the sample preparation and CF separation involves the use of guanidine-hydrochloride and a nonionic detergent (e.g., n-octyl β-D-glucopyranoside) that is compatible with the NP-RP-HPLC and ESI-oaTOF MS.

III) Mass Spectroscopic Analysis and 2-D Display Systems and Methods

In some preferred embodiments of the present invention, separated proteins are analyzed by mass spectrometry to facilitate the generation of detailed and informative 2-D protein maps. The present invention is not limited by the nature of the mass spectrometry technique utilized for such analysis. For example, techniques that find use with the present invention include, but are not limited to, ion trap mass spectrometry, ion trap/time-of-flight mass spectrometry, quadrupole and triple quadrupole mass spectrometry, Fourier Transform (ICR) mass spectrometry, and magnetic sector mass spectrometry. The following description of mass spectroscopic analysis and 2-D protein display is illustrated with ESI oa TOF mass spectrometry. Those skilled in the art will appreciate the applicability of other mass spectroscopic techniques to such methods.

In some embodiments of the present invention, ESI oa TOF mass spectrometry is used following two dimensional protein separation to provide an accurate protein separation map. For example, in one embodiments of the present invention, proteins were analyzed from human erythroleukemia (HEL) cells. The human erythroleukemia (HEL) cell line was obtained from the Department of Pediatrics at The University of Michigan. HEL cells were cultured according to the methods described in Example 1. A preparative scale Rotofor (Biorad) was used in the first dimension separation. In this experiment, 20 mg of protein was loaded. The proteins were separated by isoelectric focusing over a 5 hour period with slight modifications to the Rotofor methods described elsewhere herein. The separation temperature was 10° C., and the separation buffer contained 0.5% n-octyl β-D-glucopyranoside (OG) (Sigma), 6 M urea (ICN), 2 M thiourea (ICN), 2% β-mercaptoethanol (Biorad) and 2.5% Biolyte ampholytes, pH 3.5-10 (Biorad).

The procedure used for running the Rotofor (Rotofor Purification System, Biorad) was a modified version of the standard procedure described in the manual from Biorad. The starting power, voltage and current were 12 W, 400 V and 36 mA respectively. The ending power, voltage and current were 12 W, 1000 V and 5 mA respectively. The 20 fractions contained in the Rotofor were collected simultaneously into separate vials using a vacuum source attached by plastic tubing to an array of 20 needles which were punched through a septum. The Rotofor fractions were aliquotted in 400 μL amounts into polypropylene micro-centrifuge tubes and stored at −80° C. for further analysis as desired. The pH of the fractions was determined using pH indicator paper (Type CF, Whatman). Fractions from the Rotofor were quantified using a Bradford assay (See e.g., Wall et al., Anal. Chem., 72:1099 [2000]).

For NPS RP HPLC, separations were performed at a flow rate of 0.4 mL per minute on an analytical (3.0*33 mm) NPS RP HPLC column containing 1.5 μm C18 (ODSI) non-porous silica beads (Eichrom Technologies). The use of the 3 mm column provided more than sufficient sensitivity with the use of the LCT as well as reduced solvent consumption. The column was placed in a column heater (Timberline, Boulder Colo.) and maintained at 65° C. The separations were performed using water/acetonitrile (0.1% TFA, 0.3% formic acid) gradients. The gradient profile used was as follows: 1) 0 to 20% acetonitrile (solvent B) in 1 minutes; 2) 20 to 30% B in 2 minutes; 3) 30 to 54% B in 8 minutes; 4) 54 to 65% B in 1 minute; 5) 65 to 100% B in 1 minute; 6) 100% B in 3 minutes; 7) 100 to 5% B in 1 minute. The effective start point of this profile was one minute into the gradient due to a one-minute dwell time. The acetonitrile was 99.93+% HPLC grade (Sigma), the TFA was from 1 mL sealed glass ampules (Sigma) and the formic acid was ACS grade (Sigma). The non-ionic detergent used was n-octyl β-D-galactopyranoside (OG) (Sigma). The HPLC instrument used was a Beckman model 127s/166 and the peaks were detected on-line by a commercial ESI oa TOF/MS (LCT, Micromass, Manchester U.K.). In preferred embodiments, a detergent is used throughout the separation and detection steps that is compatible with the steps of RP HPLC and ESI oa TOF/MS (e.g., detergents of the formula n-octyl (SUGAR)pyranoside).

The ESI oa TOF/MS analyses were performed on a Micromass LCT equipped with a reflectron, a 0.5 meter flight tube and a dual micro-channel plate detector. The instrument produced protein mass spectra with a mass resolution of 5000 (FWHM). The flow from the HPLC column eluent was split to the ESI stainless steel capillary at a 1:1 ratio leaving a flow to the mass spectrometer of 0.2 mL/minute. The source temperature was held at 150° C., the desolvation temperature was 400° C., the nebulizer gas (N₂) was left at 50% maximum flow and the desolvation gas was held at 600 L/minute. The capillary voltage was held at +2500 V and the sample cone voltage was held at +45 V. The extraction cone was held at +3 V. The RF voltage was set at 1000 V with the first hexapole being biased to a positive DC offset of +7 V and the second hexapole being biased to a negative DC offset of −2 V. The detector voltage was held at 2900 V. Data was acquired for a maximum mass/charge range of 5000 resulting in a pusher cycle time of 90 μs. The data was stored to the ECP at a rate of 1 Hz and then transferred from this data-collecting computer to the main data analysis computer for generation of the data files and TIC.

Software used to analyze the mass spectra was the MaxEnt (version 1) software and Mass Lynx version 3.4 (Micromass). Typical deconvolution was performed with a wide target mass range, 1 Dalton resolution, 0.75 Da peak width and 60% peak height values. All deconvoluted mass spectra from a given TIC were added together to produce one mass spectrum for each TIC. The TIC mass spectra from each of the Rotofor fractions were then input to the 2D mapping software (available from Dr. Stephen J. Parus, University of Michigan, Department of Chemistry, 930 N. University Ave., Ann Arbor, Mich. 48109-1055).

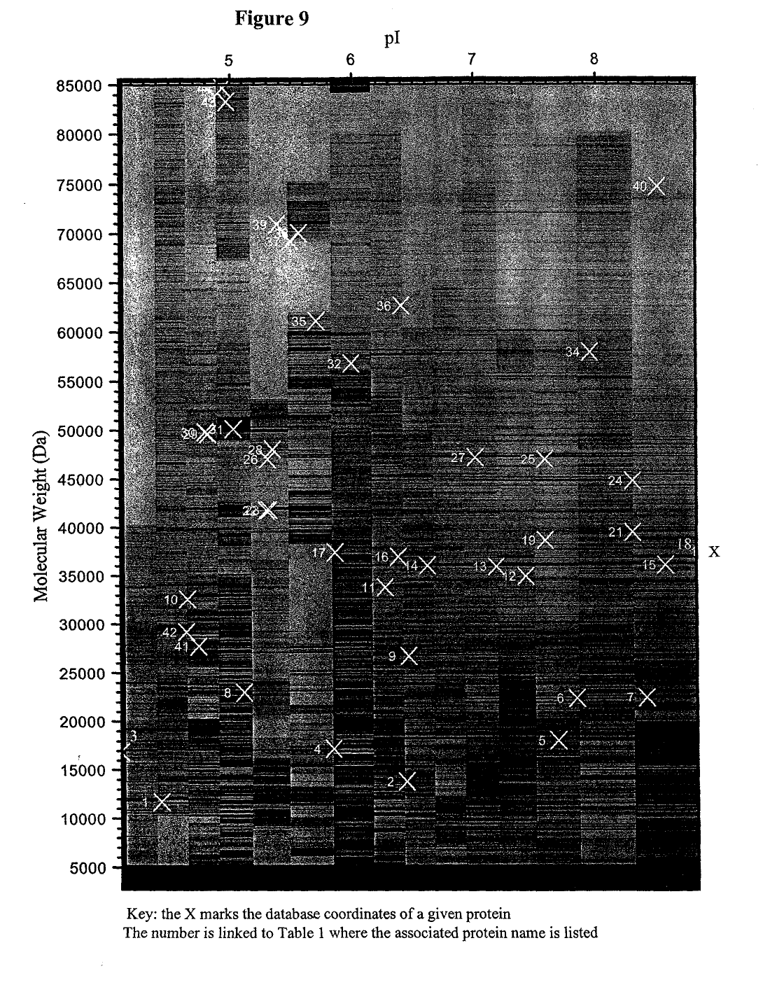

The 2-D image in FIG. 9 shows protein molecular weight in the vertical dimension and protein pI in the horizontal dimension. Individual proteins are represented as bands within the grayscale image. Protein identities were matched to this image by overlaying a virtual map of all proteins previously identified via the NPS RP HPLC separation method described above and digest analysis with MSFit database searching.

The experimental mass values were typically better than 150 to 200 parts per million of the value recorded in the SWISS-PROT database when using the Peptident database (available at http://www.expasy.ch/tools/peptident.html) to correct for possible post translational modifications. The pI could be estimated to within 0.01 to 0.5 pI units using intensity profiling as described below. Each vertical lane represents, in band format, all proteins observed via LCT mass spectral detection from the NPS RP HPLC analysis of that particular Rotofor fraction. The NPS RP HPLC separations were performed on from 17 to 60 μg of protein per Rotofor fraction. The bands in the image vary in gray scale intensity according to the intensity of the source molecular weight peaks. This image has been magnified in the intensity dimension by allowing virtual saturation of the signal of the more abundant proteins. The magnification factor is 27× or 53615/2000 (max intensity/magnification intensity). The intensity has a linear dynamic range of at least 3 orders of magnitude. Some of the same protein patterns can be seen in both the liquid phase separation and a 2D gel image from Swiss-Prot (http://expasy.cbr.nrc.ca/ch2dothergifs/publi/elc.gif). Five of the nineteen proteins identified in the 2D gel image also were found in the liquid phase separation. When comparing these images it must be kept in mind that the mass scale is linear from the liquid phase separation and logarithmic in the gel phase separation.

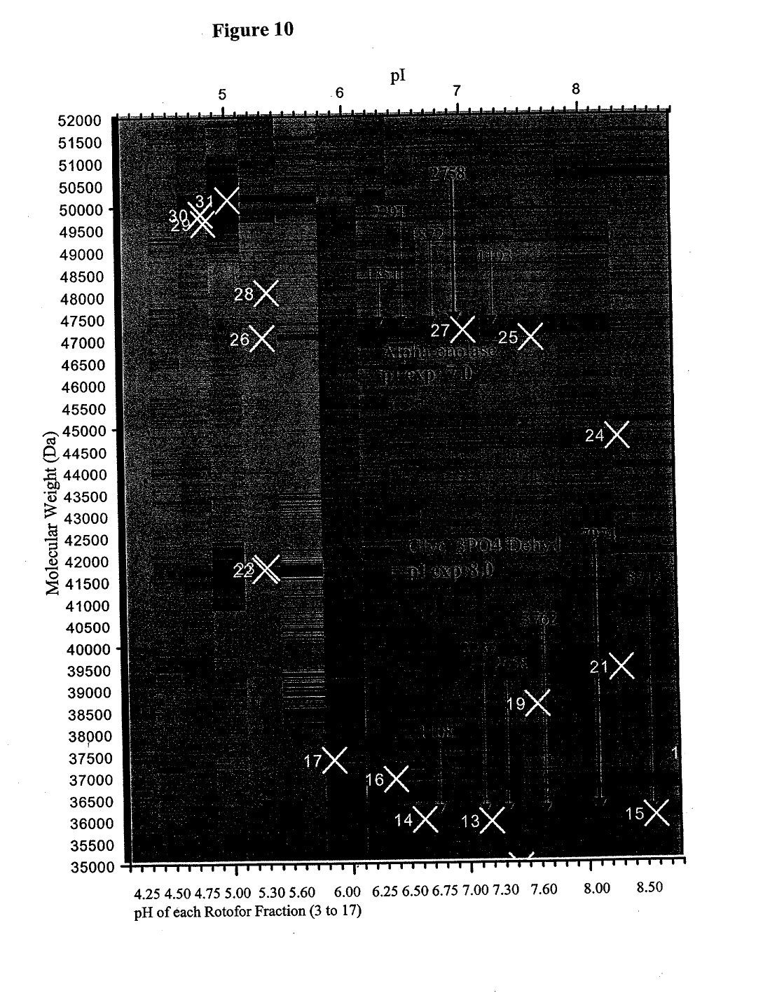

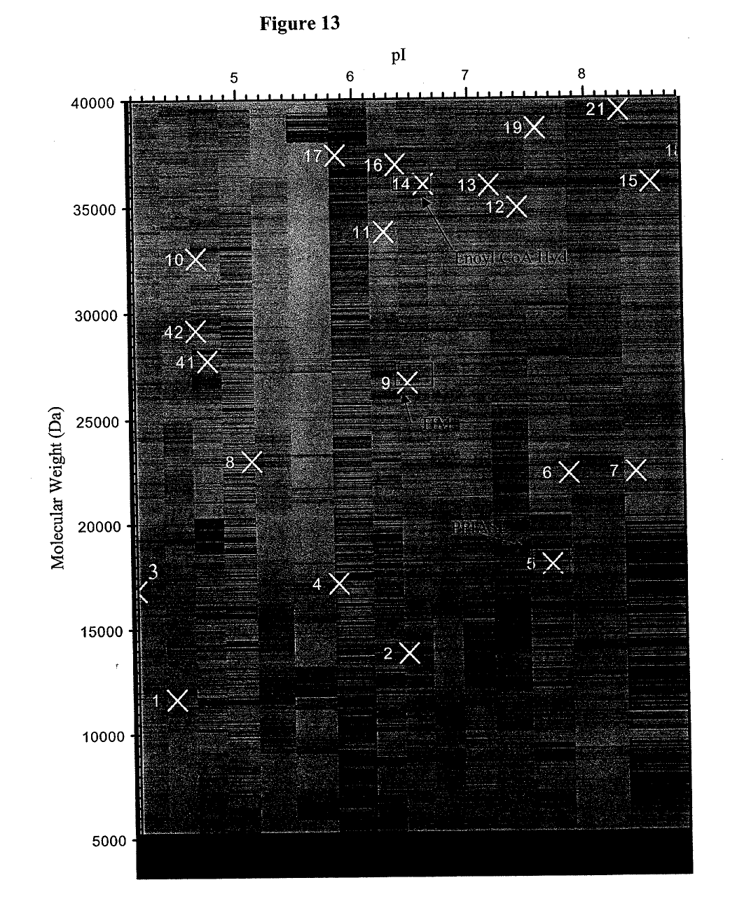

The pI of proteins isolated in the 3D liquid separation method can be estimated by observing the intensity of a given protein peak over a range of pI fractions. As a protein may spread anywhere from 2 to 6 pI fractions due to diffusion and basic cathodic drift, it should be most abundant in that fraction that is closest to its own pI. This can be observed in the zoom image of FIG. 10 (See also, zoom image of FIG. 13). Using this approach, the pI of alpha-enolase is estimated to be 7.0 (database value of 7.01), and the pI of glyceraldehyde 3-PO₄ dehydrogenase is estimated to be 8.0 (database value of 8.57). This acidic shift may be due to a post-translational modification such as phosphorylation or glycosylation.

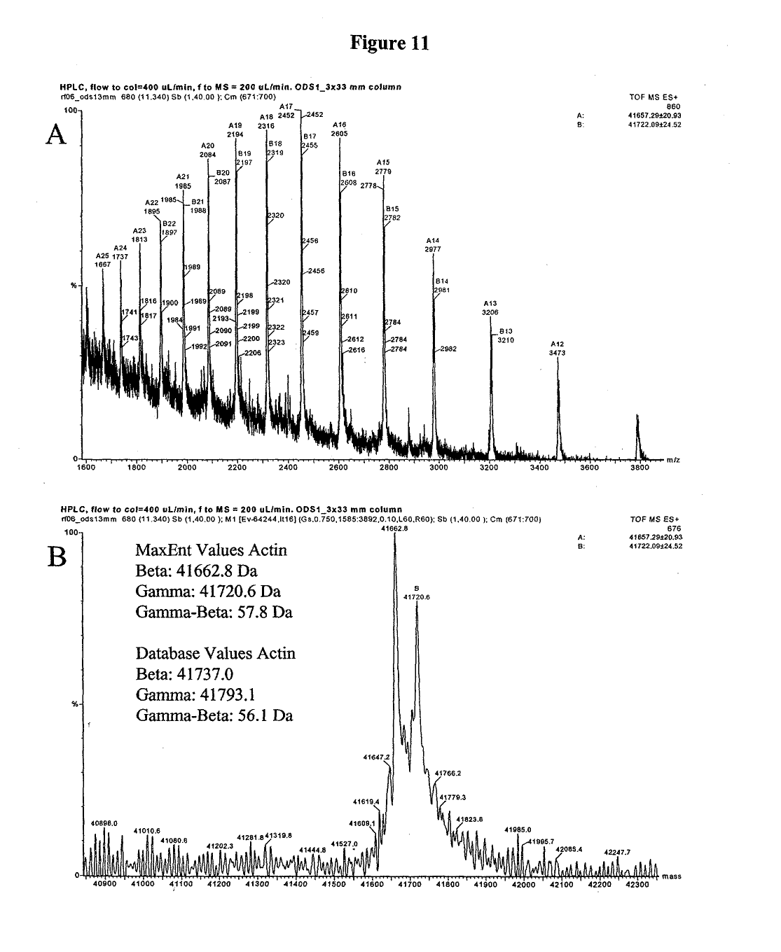

The protein molecular weights were determined by MaxEnt deconvolution of multiply charged protein umbrella mass spectra that were obtained by combining anywhere from 10 to 60 seconds of data from the initial total ion chromatogram (TIC). The umbrella for beta and gamma actin is shown in FIG. 11A, each form of actin being labeled with the charge state. FIG. 11B shows the resulting molecular weight mass spectrum for actin where the two forms of actin are separated. Note that the two forms of actin are clearly resolved from one another unlike in gel images where the actin spot always represents the co-migration of beta and gamma actin. A useful feature of the liquid phase method of the present invention is the capability of the high resolution mass spectrometry to quantitate which allows the observer to record relative levels of each form of a given protein. Consequently, it is contemplated that one cam determine the relative abundances of the phosphorylated and non-phosphorylated forms of a given protein. In addition, post-translational modifications such as phosphorylation can be found by searching the data for intervals of some integer value times 80 Da.

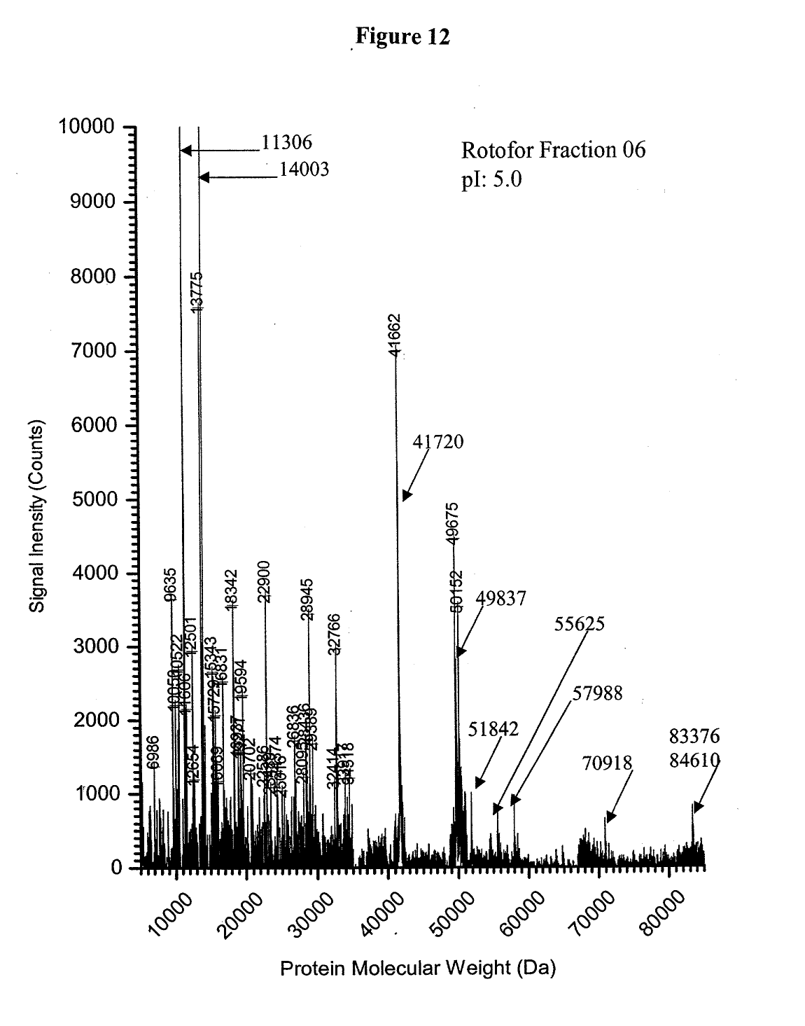

FIG. 12 shows the traditional peak view format of one of the Rotofor fraction's combined molecular weight mass spectra. All proteins were deconvoluted and then added together into one mass spectrum. There are 44 unique protein molecular weights observed in this mass spectrum. Assuming similar numbers of unique masses in all 15 of the Rotofor fractions analyzed herein, and accounting for longitudinal diffusion between fractions, it is estimated that approximately 220 unique protein masses in the image from a pI of 4.1 to a pI of 8.75. The Rotofor produces 20 fractions, though only 15 were analyzed in this work, so that around 300 unique masses should be observed in the full analysis of all Rotofor fractions. It is contemplated that lower level proteins not obtained in the above experiment can be obtained using improved HPLC gradients, 53 mm long columns and more detailed MaxEnt analyses. Using such methods, it is contemplated that the number of unique masses will be around 750.

As shown in the above experiments, the 2D protein image from the IEF-NPS RP HPLC-ESI oa TOF/MS separation of the human erythroleukemia cell lysate provides high mass resolution and high accuracy imaging of the proteins. The mass resolution allows the image to show very different forms of the same protein that have small differences in mass. With a mass resolution of 5000 Da, a 50000 Da protein can be resolved from a 50010 Da protein. Clearly, single phosphorylations on entire proteins can be observed with this level of resolution. Quantitative comparison between 2-D images can be achieved by spiking samples with known amounts of standard proteins and normalizing images through landmark proteins. Thus, the observer can detect significant abundance changes in the protein profiles of different samples. The differences can then be targeted for more detailed analysis. For example, protein bands on the image can be hyper-linked to other experimental results, obtained via analysis of that band, such as peptide mass fingerprints and MSFit search results. Thus all information obtained about a given 2-D image, including detailed mass spectra, data analyses and complementary experiments (immuno-affinity, peptide sequencing) can be accessed from the original image.

Having identified and characterized the proteins that have changed in abundance due to some disease state or drug treatment, it is possible to identify biomarkers for disease states as well as drug targets for pharmaceutical agents and monitor the presence of, or change in, such markers in a particular biological sample (e.g., tissue samples with and without exposure to a candidate drug). Indeed, drug screening and diagnostic techniques can be automated using the systems and methods of the present invention, wherein cells (e.g., experimental and control cells) are cultured, treated, and lysed using robotics and wherein the lysate is fed into the automated separation and analysis systems of the present invention.

As is clear from the above description, the methods and systems of the present invention provide a range of novel features that provide improved methods for analyzing protein expression patterns. For example, the present invention provides a combination of IEF, resulting in pI-focused proteins in liquid phase fractions, with nonporous RP HPLC and ESI oa TOF/MS to produce a 2-dimensional liquid phase protein map image analogous to that of a 2-D gel. These methods allow the identification of proteins separated by IEF-NPS RP HPLC using enzymatic digestions and mass spectrometric analysis of the resulting peptide mass fingerprints and correlation of this data with the pI and molecular of the protein found via the whole protein 3-D separation method. In some improved display embodiments of the present invention, one can view a collection of different IEF-NPS RP HPLC-ESI oa TOF/MS chromatograms in one 2-D image displaying the mass spectra in a top view protein band format, not the traditional side view peak format. The methods also allow the detection of proteins and determination of their molecular weights by analyzing the eluent from the HPLC with computational (e.g., on-line) analysis using ESI oa TOF/MS.

The IEF-NPS RP HPLC-ESI oa TOF/MS method also allows one to fully integrate and deconvolute each of the TIC's generated to display complete mass spectra of each collection of pI-focused proteins. The method also allows the display of all the integrated TIC's in one 2-D image where the vertical dimension is in terms of protein molecular weight and the horizontal dimension is in terms of protein pI. In such displays, the protein mass spectra appear as bands as they will also be viewed from the top. This image would therefore also contain relative quantitative information wherein the bands vary in intensity depending on the amount of protein present. The use of liquid phase separation techniques with the method allows for collection of protein fractions to micro-tubes or 96-well plates such that the proteins could be digested and the peptide mass maps analyzed to determine the identity of said proteins simultaneously.

IV) Automated 3D HPLC/MC Methods for Rapid Protein Characterization

In some embodiments, the present invention provides an automated system for the separation and identification of protein samples based on multiple physical properties. Accordingly, in some embodiments, the protein separation and analysis techniques described in the preceding sections are automated into one integrated, on-line system. Protein samples are separated in a first phase and a second orthogonal phase, followed by mass spectroscopy analysis. In preferred embodiments, all of the steps are automated and coordinated through an automated sample handler and a centralized control network.

Accordingly, in some embodiments, the entire separation and characterization process is controlled through one centralized control network. The network is integrated with all of the apparatus and software used for the automated process. In some preferred embodiments, the centralized control network includes a computer system. The use of a centralized control network allows for the entire separation and characterization process to be controlled from one computer terminal by one operator. The network directs sample through the appropriate separation phases. The network then controls the transfer of protein information to analysis software. The analysis software is integrated into the network and can be programmed to generate a customized report based on the information required by the user.

A. Protein Separation

As described above, the present invention provides methods for the separation of protein samples in two phases. In preferred embodiments, the methods are orthogonal, and thus allow for the generation of a two-dimensional map. In some preferred embodiments, the present invention further provides methods of automating the two phase separation.

1. Separation in a First Phase

The automated separation methods of the present invention may be used on any suitable protein sample. As discussed above, in some embodiments, the sample is solubilized in a buffer comprising a compound of the formula n-octyl SUGAR pyranoside (e.g., including, but not limited to, n-octyl β-D-glucopyransoside and n-octyl β-D-galactopyransoside).

The first dimension of the automated separation process separates proteins based on a first physical property. For example, in some embodiments of the present invention proteins are separated by charge (e.g., ion exchange chromatography). In some preferred embodiments, cation exchange chromatography is used to separate positive proteins and anion exchange chromatography is used to separate negatively charged proteins. However, the first dimension may employ any number of separation techniques including, but not limited to, ion exclusion, isoelectric focusing, normal/reversed phase partition, size exclusion, ligand exchange, liquid/gel phase isoelectric focusing, and adsorption chromatography.

In some preferred embodiments, the first separation phase is conducted in the liquid phase. In some embodiments, the first phase is ion exchange. In such embodiments, it is preferred that samples are de-salted prior to the second separation phase. In some embodiments, desalting is performed on an automated solid phase extraction (SPE) system. In some embodiments, both the ion exchange and the desalting are performed on the same automated SPE system. In other embodiments, the ion exchange is performed on a column and the eluate is directed into the automated SPE system.

In some embodiments, if proteins are present in small amounts, samples can be loaded onto the SPE columns multiple times in order to obtain a sufficient amount for analysis. Thus, the present invention has the added advantage of allowing the identification of proteins with a low level of expression. 2. Automated Sample Handling

As described in the preceding section, in preferred embodiments, samples are processed using an automated sample handling system. The present invention is not limited to any one automated sample handling system. However, in some preferred embodiments, an on-line automated, SPE system is utilized (e.g., including, but not limited to, the Prospekt automated SPE system; Spark Holland Instrumenten, The Netherlands). The advantage of on-line SPE is the direct elution of the extract from the SPE cartridge into the second phase (e.g., LC system) by the LC mobile phase. Several laborious handling steps are thus omitted, making on-line SPE much more efficient and providing superior analytical results. The superior analytical performance of on-line SPE is derived from the elimination of eluate collection, evaporation, reconstitution and injection, thus eliminating several major error sources. In addition, on-line elution transfers 100% of the purified analytes from the extraction cartridge into the LC (e.g., HPLC). This provides maximum precision and sensitivity, as well as reduced costs, thus saving solvents, glassware, and labor time. In addition, samples and SPE cartridges are processed in a completely closed system making sample tracking easy and protecting samples against light and air. It also protects the operator from contact with hazardous samples or solvents. Furthermore, less handling means fewer failures and high pressure solvent control for SPE makes the process independent of cartridge back pressure.

3. Separation in a Second Phase

In some preferred embodiments, following the first separation phase, products of the separation step are fed directly into a second liquid phase separation step. The second dimension separates proteins based on a second physical property (i.e., a different property than the first physical property) and is preferably conducted in the liquid phase (e.g., liquid-phase size exclusion). For example, in some embodiments of the present invention, proteins are separated by hydrophobicity using non-porous reversed phase HPLC (See e.g., Liang et al., Rap. Comm. Mass Spec., 10:1219 [1996]; Griffin et al., Rap. Comm. Mass Spec., 9:1546 [1995]; Opiteck et al., Anal. Biochem. 258:344 [1998]; Nilsson et al., Rap. Comm. Mass Spec., 11:610 [1997]; Chen et al., Rap. Comm. Mass Spec., 12:1994 [1998]; Wall et al., Anal. Chem., 71:3894 [1999]; Chong et al., Rap. Comm. Mass Spec., 13:1808 [1999]).

This method provides for exceptionally fast and reproducible high-resolution separations of proteins according to their hydrophobicity and molecular weight. The non-porous (NP) silica packing material used in these reverse phase (RP) separations eliminates problems associated with porosity and low recovery of larger proteins, as well as reducing analysis times by as much as one third.

In preferred embodiments, an automated on-line sample handling system utilized in the present invention fully integrates the second separation phase with the first separation step. The sample flows directly from the first phase (e.g., ion exchange) through a desalting step (e.g., SPE) to the second phase (e.g., NP-RP HPLC). In preferred embodiments (e.g., those utilizing the Prospekt system) the HPLC column is integrated into the automated sample handling system. For example, a multi valve system can be utilized where valve-switching is used to bring the extraction cartridge into the HPLC system. In some embodiments, a sample is passed through the second phase separation step (e.g., NP-RP HPLC) greater than one time (e.g., twice) in order to improve selectivity and resolution. For example, in some embodiments, two different NP-RP-HPLC columns are utilized in tandem. The automation of protein separation increases efficiency and speed as well as decreases sample loss or potential contamination that may occur through handling.

B. Protein Identification by Mass Spectroscopy

Following separation in the first and second phase, the automated sample handling system transfers samples to the mass spectroscopy step. The present invention is not limited to any one mass spectroscopy technique. Indeed, a variety of techniques are contemplated. For example, techniques that find use with the present invention include, but are not limited to, ion trap mass spectrometry, ion trap/time-of-flight mass spectrometry, quadrupole and triple quadrupole mass spectrometry, Fourier Transform (ICR) mass spectrometry, and magnetic sector mass spectrometry. In preferred embodiments, the MS analysis is automated and is performed on-line. In some embodiments, the eluent from the second separation phase is split into two fractions. A fraction of the effluent is used to determine molecular weight by either MALDI-TOF-MS or ESI oa TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI oa TOF. The molecular weight 2-D protein map is matched to the appropriate digest fingerprint by correlating the molecular weight total ion chromatograms (TIC's) with the UW-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

A detailed discussion of the use of 3-D maps generated by the automated separation process of the present invention to identify and characterize proteins is provided in the above sections. In some embodiments, the present invention provides a 3-D map in which the first dimension represents a first physical property (e.g., charge or isoelectric point), the second dimension represents a second physical property (e.g., hydrophobicity or molecular weight), and the third dimension represents the molecular weight and relative abundance of proteins present in the sample. In some embodiments, the data from the 3-D protein map is used to search protein data bases in order to determine the identity of the proteins.

In some embodiments of the present invention, sample analysis is automated and integrated with the centralized control network. For example, mass spectroscopy data is transferred to an integrated computer system containing software for the generation of 3-D protein maps. The integrated computer system is also capable of searching databases and generating a report. The report is provided to the operator in a format that is customized to the particular application. For example, if an experiment was designed to identify unknown components of a solution, the report identifies components of the 3-D map as particular proteins. Conversely, if an experiment is designed to compare the protein expression profiles of two samples, the report may identify proteins that are present in one sample and absent in another or are present at different abundances between the two samples.

C. Automated Protein Separation and Characterization in Practice

Illustrative Example 8 describes one particular embodiment of the present invention where an automated on-line Prospekt system was used to separate a protein sample based on charge and hydrophobicity. Siberian Permafrost whole cell lysate was first separated using a mini MonoQ anion exchange column. A graph of the Mini Q column eluent is shown in FIG. 17. Fractions (1 minute each) from the anion exchange column gradient were fed directly into the second step using the automated Prospekt system. The Prospekt then trapped the fractions on 10 C4 SPE cartridges. Each cartridge was washed with the reverse-phase HPLC starting buffer to remove residual salt. The Prospekt system integrates the HPLC and SPE steps with a multi valve switching system. Following the wash step, the eluent from the SPE cartridge was directly transferred to the NP-RP HPLC column.

The fractions were separated using a tandem column method. A gradient was applied to the HPLC column. The HPLC column was then switched back to the initial buffer and allowed to equilibrate. The eluent from the first gradient is then passed through a second (different) HPLC column. The use of a second tandem column increases resolution and selectivity. This step is repeated for each of the SPE cartridges (each representing one anion exchange fraction).

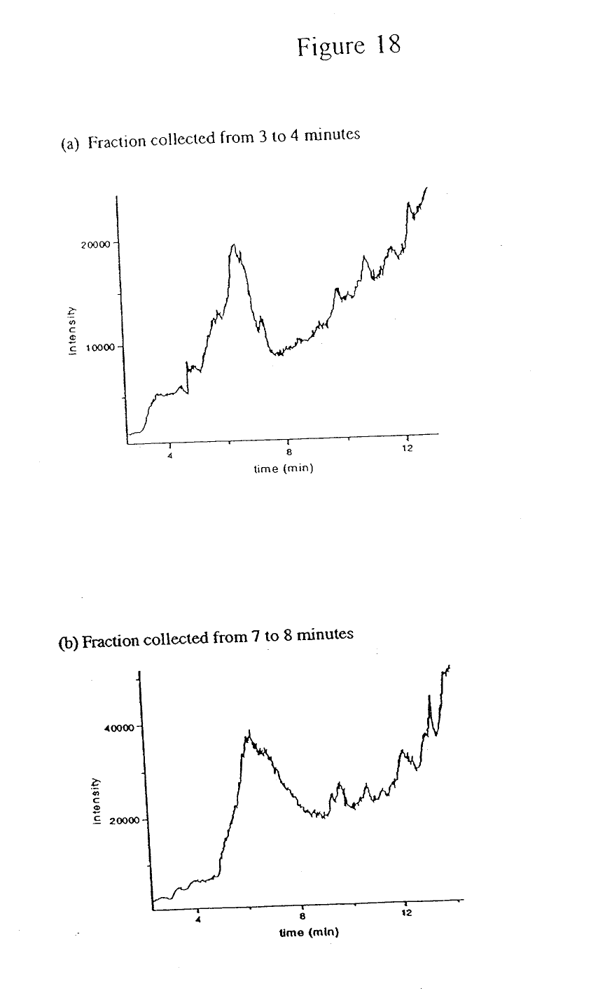

Following separation by NP-RP-HPLC, protein fractions were analyzed online by MS to determine their molecular weight and abundance. The eluent from the column was split into two fractions. One fraction is digested enzymatically before MS. Both the digested and non-digested sample were analyzed by ESI oa TOF TIC (total ion count) mass spectroscopy. Total ion count profiles are shown in FIGS. 18A and 18B.

V) Differential Display Analysis of Protein Maps

In some embodiments, the present invention provides a multi-dimensional differential display map of a multi-phase protein separation. In some embodiments, proteins from two different cell types (e.g., cancerous and non-cancerous cells, differentiated and undifferentiated, drug treated and non drug treated) are separated in two or more (e.g., three) dimensions and a high-resolution digital image is generated that displays the differences in protein abundance between the two cell types.

This three dimensional separation method of the present invention allows for the creation of a protein map image that shows, for example, the pI and molecular weight. The end result is a high-resolution digital image showing a complex pattern of proteins separated by pI and molecular weight and indicating relative protein abundances. In some embodiments, two images are created for different cell types (e.g., cancerous and non-cancerous cells or two different cancerous cells), and one image is subtracted from the other, creating a “differential display” that shows the differences between the two cell types. The differential display shows if a protein is present in differing amounts in the two cell types, or if proteins are present in one cell type and absent in the other. As described in greater detail below, in some embodiments, proteins of interest are identified simultaneously with the determination of protein mass performed in the third dimension ESI-oaTOF/MS by splitting off the eluant from the 2° dimension HPLC separation and performing proteolytic digestion on the collected fractions.

The methods described below for identifying proteins that are present in differing amounts between two or more cell types (e.g., cancerous and non-cancerous cells) find utility in the rapid diagnosis of cancers and disease states in individuals. In addition, in some embodiments, the methods of the present invention allow for the tailoring of drug therapies and treatments for affected individuals based on their protein profiles (e.g., of their cancer tissues).

For example, in some embodiments, Isoelectric Focusing/Nonporous Silica High Performance Liquid Chromatography/Electrospray Ionization-orthogonal extraction Time of Flight Mass Spectrometry (IEF/NPS HPLC/ESI-aaTOF/MS) is used to separate proteins based on isoelectric Point (pI), hydrophobicity and mass to charge ratio. Methods for such separations are described in Examples 8 and 9 and the above sections. The present invention is not limited to the separation and detection methods described below. Any suitable methods may be utilized, including but not limited to, those disclosed in the preceding description and the illustrative examples below.

A. Protein Separation and Detection

In some embodiments, proteins from two or more cell types are separated in first and second dimensions. In some embodiments, the first separation dimension is isoelectric focusing, which separates proteins based on isoelectric point (pI). Any suitable method may be utilized for isoelectric focussing, including but not limited to, Rotofor (Biorad), carrier ampholyte based slab gel IEF separation and harvesting with a whole gel eluter (WGE), and IPG slab gel IEF separation and harvesting with a whole gel eluter (WGE). Methods for performing such separations are described in Example 9 below.

In some embodiments, following separation in a first dimension, samples are separated in a second dimension by non-porous RP HPLC (See Example 9). In preferred embodiments, the NP RP HPLC methods utilized in the present invention allow for rapid, near-baseline separations of proteins by reversed phase HPLC with high recovery of the proteins. Excellent separations are important so that when proteins are collected as fractions, then digested by proteolytic enzymes and analyzed by mass spectrometry, the peptide masses submitted to the MS-Fit database represent only one or a few proteins at most. This increases the likelihood of an accurate match for protein identification. High recovery is important to ensure that enough protein is collected to allow for mass spectrometric detection of the digested protein fragments.

In some embodiments, the proteins that elute from the second separation dimension (e.g., NP RP HPLC separation) are analyzed by mass spectrometry to determine their molecular weight and identity. For this purpose the eluant from the HPLC column is split. One portion of the eluant is connected on-line to an Electrospray Ionization orthogonal acceleration Time of Flight Mass Spectrometer (ESI oa TOF-MS.) The other portion is split off to a UV-Vis detector, followed by an auto collector where the proteins are collected in accordance with their peak profile from the UV-Vis detector. These proteins are digested by proteolytic enzymes, and the mass of the resulting fragments is determined by either Matrix Assisted Laser Desorption Ionization Mass Spectrometry (MALDI-MS) or ESI oa TOF-MS. The peptide masses, along with the pI and molecular weight of the protein determined in previous parts of the experiment, are submitted to a database such as Ms-Fit for protein identification.

B. Chromatogram Deconvolution

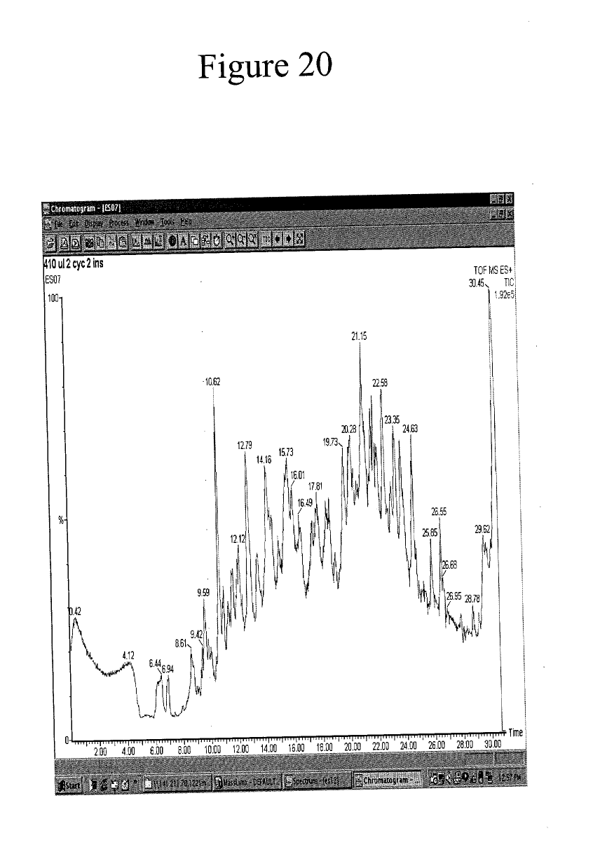

In some embodiments, following mass spectroscopy, the mass spectrum is deconvoluted to generate the mass of protein peaks (See Example 9). The ESI-oaTOF/MS provides the data from its detector in two modes, a Mass Spectrum and a Total Ion Chromatogram. The mass spectrum is a snapshot of all of the masses in the relevant range that are hitting the detector in one cycle. The TIC is a measure of all of the ions hitting the MS detector over the course of the HPLC run. As proteins are eluted from the HPLC and hit the MS detector, they appear as peaks in the TIC (see FIG. 20).

When an electrospray source is used to ionize proteins, the proteins become multiply charged, and several charge states may be present at one time. The resulting mass spectrum looks like an umbrella, with many peaks representing the same protein (see FIG. 19). Traditional methods of deconvolution using commercial software generate the actual mass of the protein and the relative abundance of the protein based on the abundances of all of the multiply charged protein peaks.

However, in preferred embodiments, the novel methods of the present invention are used to sum mass spectra from the TIC. The methods of the present invention allow for the detection of lower abundance proteins amongst the higher abundance proteins. In some embodiments, the methods of the present invention comprise manually looking at mass spectrum (e.g., 0.95 seconds of data at a time) to determine when each protein starts and stops, and summing only the spectra that contain the protein of interest. This increases the signal to noise for lower abundance proteins, because the noise from flanking cycles is not added to the summed mass spectrum. In other embodiments, the summing method is automated (e.g. with a computer software program and a computer processor).

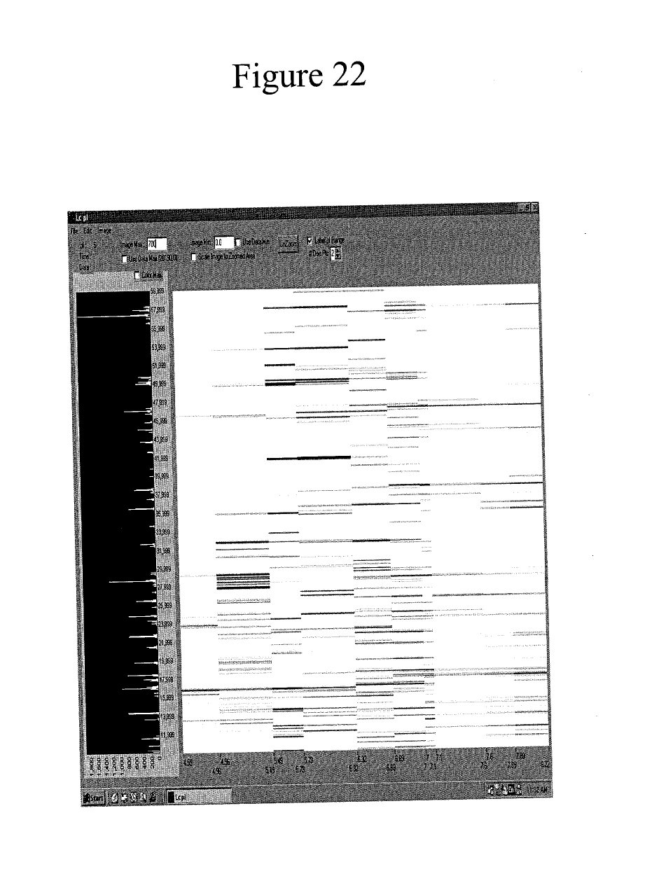

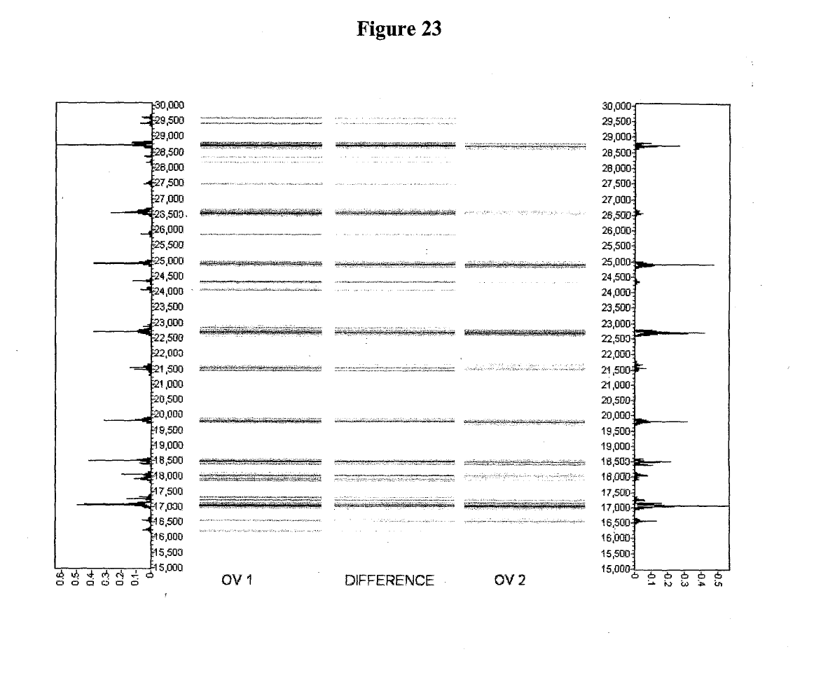

In some embodiments, once all of the regions that contain protein are determined and the deconvolution performed for each protein, the deconvoluted mass spectra are saved as text files. The text files for all of the proteins from one pI fraction are summed and they are displayed in 2-D plot in which the peaks are displayed in a “banding pattern” much like they are in gels (i.e., each band represents one protein). In the 2-D plot, the x axis is pI, the y axis is mass, and the intensity (corresponding to the abundance of the particular protein) of each band in the mass spectrum is converted to 256 color gray scale, so bands appear in a gradient of blacks and grays against a white background (see FIG. 22). Several or all of the pI fractions may be placed side by side in this manner to view the entire pI vs. mass plot for the sample.

C. Differential Display

In some embodiments, differences between deconvoluted mass spectrums are viewed as digital images. In some embodiments, the present invention provides computer software programs for the subtraction and differential display of 2-D protein maps of two or more cell types (e.g., cancerous cells and non-cancerous cells). In some embodiments, a point by point subtraction for each data point is performed and differences are represented in two colors (See FIG. 23 for one illustrative example). Bands corresponding to each cell line are represented by one color. In the subtracted map (shown in the center of FIG. 23), proteins that are present in one cell type but not the other appear as bands of the color corresponding to their cell type. Proteins that are present in both samples, but at a different abundance are shown in a lighter version of their color (due to the subtraction of a band of lesser intensity from one of greater intensity or vice-versa). Proteins present at a similar abundance are represented by a dim band (due to the subtraction of colors of a similar intensity). The two color representation thus provides information on the presence or absence of proteins in one sample but not the other as well as the relative abundance of proteins present in both samples.

In other embodiments, differences are presented as two distinct color gradients, with each color gradient corresponding to proteins of one cell type. Such a method is advantageous for observing small differences in data points that appear as a dim color in the two color plot (e.g., data points corresponding to proteins present at similar abundances in the two samples). Each color is bright and differences are indicated by a different color. However, no distinction is possible between cases of non-zero difference due to protein abundance in both cell lines and non-zero difference due to a given band existing in one cell line but not the other.

Accordingly, in some embodiments, in order to optimize the display of both the presence or absence of a protein as well as differences in abundance on one display, a four-color scheme is employed. For example, a four color mapping scheme is used if one wishes to tell if a protein exists in the difference map because the other cell line does not any contain protein at all at that location or because the other cell line contains less (or more) protein at that location. Two of the four colors are used when proteins are present in both cell lines with the specific color indicating which proteins are more abundant. The other two colors are used when one cell line had no protein present. In all four cases, the intensity of the colors represent the difference magnitude (and the color hue the type of difference). Such a differnce has potential biological relevance. For example, the four color scheme is able to inform the user that a given protein is present in both cell lines, but the quantity changed. The other case, where the protein is not present in one cell line, could mean it had been altered and was now appearing at that new position, or all of it had been changed and was no longer present. As an example, in FIG. 23, both cell lines contain some protein at 26,500 Daltons. The left OV1 image contains more protein than the right OV2 image and so the difference is colored in the color corresponding to OV1. At 27,500 Daltons, OV1 has protein but OV2 does not. In the two color scheme, the difference is again colored in the color corresponding to OV1. In a four color scheme, the difference is colored a third color to indicate that OV1 is more intense because OV2 is lacking that particular color. A fourth color indicates that, for example the color OV1 is more intense because it is present in a greater abundance.

In still other embodiments, the software allows a user to select the options of displaying either a map that depicts changes in abundance, or a map that shows when a cell line lacks a protein (e.g., indicating the disappearance of a protein, the appearance of a new protein, or a protein pI shift). The present invention is not limited to the representations described herein. Any representations that shows the subtraction of proteins present in one or more samples may be utilized.

In some embodiments, the high mass resolution of the method of the present invention utilize computer video display technology. With 100,000 data points per mass spec and typically only 1000 computer video screen pixels onto which to display them, data from 100 points must be represented at one video monitor location. When displayed as an image, only the maximum, average, or mean value within that 100-point data range is shown. For a difference plot, it is possible that within a 100-point subset, some points may have the first cell line more abundant than the second and vice-versa. Besides differences in abundance, the presence of new or shifted proteins in one cell line is an important feature to identify. Such proteins may fall within the 100 data point display resolution and would not be depicted if other larger differences existed that would instead be shown. While the display could be zoomed so that at least one pixel was used per data point, it would not be apparent from the overall view where exactly to zoom. Accordingly, in some embodiments, the present invention provides approaches to aid in detecting sub-features. For example, in some embodiments, as each sub-region is calculated, it is analyzed for small peaks and a list produced for examination in greater detail. Alternatively, in other embodiments, a second zoomed plot with higher pixel resolution is used to show a subregion of the overall data display and have it track a cursor in that main display. In some embodiments, the present invention provides algorithms to decrease the time to plot multiple points onto one pixel. Reducing the display generation time is desirable since much zooming to examine sub-regions is performed.

In the methods described above, differences are presented as an image, permitting rapid visual assimilation of cell line changes. In alternative embodiments, the present invention provides analysis of differences between cell lines by overlaying the multiple individual x-y (m/z vs. intensity) line plots. However, in preferred embodiments, an intermediate approach is utilized to display x-y line plots of the differences between cell lines. The plots are arranged vertically along the mass axis and are side-by-side at their corresponding pI location. There are two differences between this method and display as an image. Rather than using color intensity or specific color to represent the difference magnitude, the length of the plotted line is used. Secondly, both positive and negative differences can be shown at each m/z value by drawing a line both left and right of the center zero difference value.

D. Applications of Differential Display

The differential display maps of the present invention find use in a variety of situations where comparison of two samples is desired (e.g., comparison of two cell samples). An image generated by the methods of the present invention represents the data in a form visually similar to what is physically obtained by commonly used 2-D slab gel techniques. The methods of the present invention described above have several advantages over the presently available gel methods. For example, the resolution is significantly higher at 1 Dalton over a range of 100,000. Gel resolution is determined by gel characteristics, band spreading and video resolution when digitizing the gel image. Gel lanes may exhibit curvature, distortion, non-linearity, etc. Such errors may be inconsistent between two sample runs (e.g., in the case of differential display techniques). Attempts to correct for errors involve algorithms that involve changing the raw data. The mass spec technique of the present invention suffers from none of these limitations. For example, the methods of the present invention produces data containing high mass resolution to allow for the detection of small m/z shifts and do not require corrections that involve altering the raw data. Traditional gel methods do not.

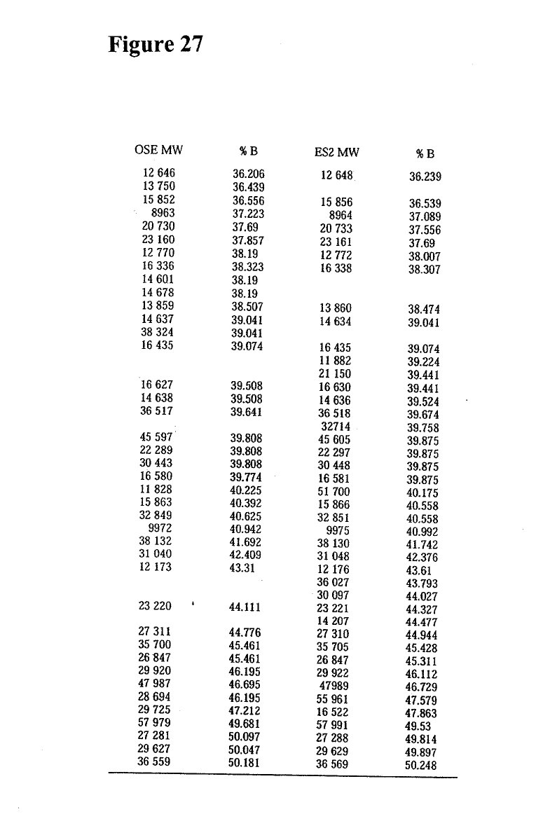

The use of the three-parameter separation and characterization methods of the present invention are useful in cases in which the proteins cannot be readily identified by peptide mapping methods and database searching (e.g., because of similar molecular weights). This is shown in FIG. 27, which lists the MW values of proteins in fraction 6 that have not been identified by peptide mapping. The liquid phase separation technique described herein provides a third parameter for matching unknown proteins from different sources. For example, in some embodiments, proteins are matched on the basis of their hydrophobicities.

The highly accurate methods of the present invention make them suitable for a number of applications. For example, in some embodiments, the methods of the present invention are used to compare two cell types (e.g., cancerous and non-cancerous cells). Such methods are used to diagnose diseases such as cancer, to determine a stage or type of a particular cancer or tumor, and to monitor progression or remission of a disease stage (e.g., cancer). Information gathered from the differential display maps of the present invention is used to provide a prognosis to a patient, as well as to determine an appropriate treatment (e.g., to determine whether or not to provide a specific chemotherapy agent).

In some embodiments, any or all of the three images (e.g., the two master images and the differential display image) are linked (e.g., through hyperlinks) to a database containing the numerical data that was used to create each image (e.g., pI, abundance, LC retention time and molecular weight), as well as the results of the proteolytic digestion of the protein. In preferred embodiments, such a database is searchable so that a user who is looking at an image created from a particular cell line (e.g., a particular cancer cell line) and is interested in a particular protein in the image, could then search other databases to find out if a protein with the same pI, molecular weight and/or retention time occurs, for example, in a different cell line (e.g., a different cancer cell line or different stage of the same cancer).

In some embodiments, protein profiles are correlated with information on prognosis of patients having a particular profile and the response of subjects with a particular profile to a given treatment. Hyperlinks imbedded in each profile provide access to any available information. Such information aids the clinician or researcher in their ability to provide a prognosis or determine the optimum treatment for a particular patient, thus allowing the personalization of treatment.

In some embodiments, databases containing protein profiles and differential display images are located on an Internet server. In preferred embodiments, the server is connected to the world wide web, allowing individuals located world-wide to obtain access to information. In some embodiments, users add protein profiles and differential display maps, as well as the underlying information, to the database, thus increasing the available information and improving correlations to clinical information.

Experimental

The following examples serve to illustrate certain preferred embodiments and aspects of the present invention and are not to be construed as limiting the scope thereof.

### EXAMPLE 1

### HEL Cell Sample Preparation

The human erythroleukemia (HEL) cell line was obtained from the Department of Pediatrics at The University of Michigan. HEL cells were cultured (7% CO₂, 37° C.) in RPMI-1640 medium (Gibco) containing 4 mM glutamine, 2 mM pyruvate, 10% fetal bovine serum (Gibco), penicillin (100 units per mL), streptomycin (100 units per mL) and 250 mg of hygromycin (Sigma). The HEL cell pellets were washed in sterile PBS, and then stored at −80° C. The cell pellets were then re-suspended in 0.1% n-octyl β-D-galactopyranoside (OG) (Sigma) and 8 M urea (Sigma) and vortexed for 2 minutes to effect cell disruption and protein solubilization. The whole cell protein extract was then diluted to 55 mL with the Rotofor buffer and introduced into the Rotofor separation chamber (Biorad).

### EXAMPLE 2

### 1-D Gel and SDS PAGE Separation

HEL cell proteins, resolved by Rotofor separation into discrete pI ranges, were further resolved according to their apparent molecular weight by SDS-PAGE. This procedure takes approximately 14 hours to complete. Samples of rotofor fractions were suspended in an equal volume of sample buffer (125 mM Tris (pH 6.8) containing 1% SDS, 10% glycerol, 1% dithiothreitol and bromophenol blue) and boiled for 5 min. They were then loaded onto 10% acrylamide gels. The samples were electrophoresed at 40 volts until the dye front reached the opposite end of the gel. The resolved proteins were visualized by silver staining. The gels were fixed overnight in 50% ethanol containing 5% glacial acetic acid, then washed successively (for 2 hours each) in 25% ethanol containing 5% glacial acetic acid, 5% glacial acetic acid, and 1% glacial acetic acid. The gels were impregnated with 0.2% silver nitrate for 25 min. and were developed in 3% sodium carbonate containing 0.4% formaldehyde for 10 min. Color development was terminated by impregnating the gels with 1% glacial acetic acid, after which the gels were digitized.

### EXAMPLE 3

### 2-D PAGE

In order to prepare protein extracts from the HEL cells, the harvested cell pellets were lysed by addition of three volumes of solubilization buffer consisting of 8 M urea, 2% NP-40, 2% carrier ampholytes (pH 3.5 to 10), 2% β-mercaptoethanol and 10 mM PMSF, after which the buffer containing the cell extracts was transferred into microcentrifuge tubes and stored at −80° C. until use.

Extracts of the cultured HEL cells were separated in two dimensions as previously described by Chen et al. (Chen et al., Rap. Comm. Mass Spec. 13:1907 [1999]) with some modifications as described below. Subsequent to cellular lysis in solubilization buffer, the cell lysates from approximately 2.5×10⁶ cells were applied to isoelectric focusing gels. Isoelectric focusing was conducted using pH 3.5 to 10 carrier ampholytes (Biorad) at 700 V for 16 h, followed by 1000 V for an additional 2 hours. The first dimension tube gel was soaked in a solution of 2 mg/mL of dithioerythritol (DTE) for 10 minutes, and then soaked in a solution of 20 mg/mL of iodoacetamide (Sigma) for 10 minutes, both at room temperature. The first-dimension tube gel was loaded onto a cassette containing the second dimension gel, after equilibration in second-dimension sample buffer (125 mM Tris (pH 6.8), containing 10% glycerol, 2% SDS, 1% dithioerythritol and bromophenol blue). For the second-dimension separation, an acrylamide gradient of 11.5% to 14% was used, and the samples were electrophoresed until the dye front reached the opposite end of the gel. The separated proteins were transferred to an Immobilon-P PVDF membrane. Protein patterns in some gels were visualized by silver staining or by Coomassie blue staining, and on Immobilon-P membranes by Coomassie blue staining of the membranes.

### EXAMPLE 4

### Rotofor Isoelectric Focusing

A preparative scale Rotofor (Biorad) was used in the first dimension separation. This device separated the proteins in liquid phase according to their pI, and is capable of being loaded with up to a gram of protein, with the total buffer volume being 55 =mL. Alternatively, for analysis of smaller quantities of protein, a mini-Rotofor with a reduced volume can be used. These proteins were separated by isoelectric focusing over a 5 hour period where the separation temperature was 10° C. and the separation buffer contained 0.1% n-octyl β-D-galactopyranoside (OG) (Sigma), 8 M urea (ICN), 2% β-mercaptoethanol (Biorad) and 2.5% Biolyte ampholytes, pH 3.5-10 (Biorad). The procedure used for running the Rotofor (Rotofor Purification System, Biorad) was of the standard procedure described in the manual from Biorad as modified herein. The 20 fractions contained in the Rotofor were collected simultaneously, into separate vials using a vacuum source attached by plastic tubing to an array of 20 needles, which were punched through a septum. The Rotofor fractions were aliquotted into 400 μL amounts in polypropylene microcentrifuge tubes and could be stored at −80° C. for further analysis if necessary. An advantage of gel methods is the ability to store proteins stably in gels at 4° C. for further use. The concentration of protein in each fraction was determined via the Biorad Bradford based protein assay. The pH of the fractions was determined using pH indicator paper (Type CF, Whatman).

### EXAMPLE 5

### NP RP HPLC

Separations were performed at a flow rate of 1.0 mL/minute on an analytical (4.6*14 mm) NP RP HPLC column containing 1.5 μm C18 (ODSI) non-porous silica beads (Micra Scientific Inc.). The column was placed in a Timberline column heater and maintained at 65° C. The separations were performed using water/acetonitrile (0.1% TFA, 0.05% OG) gradients. The gradient profile used was as follows: 1) 0 to 25% acetonitrile (solvent B) in 2 minutes; 2) 25 to 35% B in 2 minutes; 3) 35 to 45% B in 5 minutes; 4) 45 to 65% B in 1 minute; 5) 65 to 100% B in 1 minute; 6) 100% B in 3 minutes; 7) 100 to 5% B in 1 minute. The start point of this profile was one minute into the gradient due to a one-minute dwell time. The acetonitrile was 99.93+% HPLC grade (Sigma) and the TFA were from 1 niL sealed glass ampules (Sigma). The non-ionic detergent used was n-octyl β-D-galactopyranoside (OG) (Sigma). The HPLC instrument used was a Beckman model 127s/166. Peaks were detected by absorbance of radiation at 214 nm in a 15 μL analytical flow cell.

Protein standards (Sigma) used as MW protein markers and for correlation of retention time, molecular weight and hydrophobicity were bovine serum albumin (66 kDa), carbonic anhydrase (29 kDa), ovalbumin (45 kDa), lysozyme (14.4 kDa), trypsin inhibitor (20 kDa) and oc-lactalbumin (14.2 kDa).

### EXAMPLE 6

### MALDI-TOF MS of NP RP HPLC Isolated Proteins

The MALDI-TOF MS analyses were performed on a Perseptive Voyager Biospectrometry Workstation equipped with delayed extraction technology, a one-meter flight tube and a high current detector. The N₂ laser provided light at 337 nm for laser desorption and ionization. MALDI-TOF MS was used to determine masses of peptides from protein digests using a modified (described herein) version of the two layer dried droplet method of Dai et al. (Dai et al., Anal. Chem., 71:1087 [1999]). The MALDI matrix α-cyano-4-hydroxy-cinnamic acid (α-CHCA) (Sigma Chemical Corp., St Louis, Mo., USA) was prepared in a saturated solution of acetone (1% TFA). This solution was diluted 8-fold in the same acetone solution (1% TFA) and then added to the sample droplet in a 1:2 ratio (v:v). The mixed droplet was then allowed to air dry on the MALDI plate prior to introduction into the MALDI TOF instrument for molecular weight analyses.

The proteins were collected into 1.5 mL polypropylene micro-tubes containing 20 μL of 0.8% OG in 50% ethanol. In preparation for enzymatic digestion the acetonitrile was removed via speedvac at 45° C. for 30 minutes. A solution of 200 mM NH₄HCO₃ (ICN)/1 mM β-mercaptoethanol was then added in a 1 to 2 ratio to the remaining solution in the tubes, resulting in a solution of 50 to 100 mM NH₄HCO₃ with a total volume of approximately 150 μL. Subsequently 0.25 μg of enzyme was added to this solution and then the mixture was vortexed and placed in a 37° C. warm room for 24 hours. The enzymes used were either trypsin (Promega, TPCK treated), which cleaves at the carboxy side of the arginine and lysine residues, or Glu-C (Promega), which in 50-100 mM NH₄HCO₃ solution cleaves at the carboxy side of the glutamic acid residues.

The digest solutions were typically 100 μL in volume and 30 to 50 μL of this solution was desalted and concentrated to a final volume of 5 μL using Zip-Tips (Millipore) with 2 μL C 18 resin beds. The purified peptide solution was then used to spot onto the MALDI plate for subsequent MALDI-TOF MS analysis. All spectra were obtained with 128 averages and internally or externally calibrated using the PerSeptive standard peptide mixture containing angiotensin I, ACTH(1-17), ACTH(18-39) and ACTH(7-38) (PerSeptive Biosystems).

These digests were then used to aid in the identification of the proteins by MALDI-TOF MS analysis and MSFit database searching (Wall et al., Anal. Chem., 71:3894 [1999]). The peptide mass maps were searched against the Swiss and NCBInr protein databases using MSFit allowing for 2 missed cleavages. The molecular weight ranged from 5 kDa to 70 kDa and the pI ranged over the full pI range. Externally calibrated peptide masses were searched with 400 ppm mass accuracy and internally calibrated peptide masses were searched with 200 ppm mass accuracy.

### EXAMPLE 7

### Chromatofocusing

In one exemplary embodiment of the chromatic focusing techniques of the present invention, proteins are extracted from cells using chemical lysing procedure. The lysis buffer consists of 6M guanidine-hydrochloride, 20 mM n-octyl β-D-glucopyranoside and 50 mM Tris. Cells are vortexed rigorously and kept overnight at −20° C. They are subsequently centrifuged at 17,000 rpm for 20 min. The supernatant is removed from the cell debris and re-centrifuged at high speed to further remove any particulate. For the best reproducible results, lysate is best used within 48 hrs. Buffers for this CF are (A) Imidazole-HAC, 0.1% guanidine-hydrochloride, 0.05% n-octyl β-D-glucopyranoside, pH 7.2, and (B) Polybuffer 74 (diluted 1:10), 0.1% guanidine-hydrochloride, 0.05% n-octyl β-D-glucopyranoside, pH 4. The CF column in this example is Mono P HR 5/20 (Amersham Pharmacia, Uppsala, Sweden) with a flowrate of 1 mL/min at room temperature. Prior to injection lysate is equilibrated with buffer A with a loading time of 20 min. The sample loadability for this CF column is 10 mg of protein. The separation profile is monitored at 280 nm while the pH gradient is monitored using a pH flowcell meter, also from Amersham Pharmacia.

The CF column is equilibrated with buffer A to define the upper pH range (7 in this case) of the pH gradient. The second “focusing” buffer B is then applied to elute bound proteins, in the order of their isoelectric (pI) points. The pH of buffer B is 4, which defines the lower limit of the pH gradient. The pH gradient is formed as the eluting buffer B titrates the buffering groups on the ion-exchanger.

The pI-focused liquid fractions from CF are analyzed in the second dimension using NP-RP-HPLC. Non-porous RP-HPLC columns (Eichrom Technologies, Darien, IL, USA) are used as the second orthogonal separation dimension after CF in order to obtain a 2-D protein map that is capable of competing with 2-D gel. These columns are excellent for protein separation due to their high protein recovery, speed and efficiency. To achieve optimal protein separation, the columns should be kept at a high temperature (e.g., 60° C.). This elevated temperature also improves selectivity. Selectivity as well as resolution can also be enhanced by using multiple NP columns in series. RP-HPLC columns packed with non-porous silica beads (Eichrom Technologies) such as ODS1, 2 and 3 are all well suited for these tasks.

Proteins that elute from NP-RP-HPLC separation can be directly analyzed by MS to determine their molecular weight, identity and relative abundance. In this case the eluted proteins are sized simultaneously by ESI-oaTOF MS (LCT, Micromass, Manchester, UK). The other part of the eluted proteins from the split valve can be collected using a fraction collector for enzymatic digestion to obtain peptide maps with a MALDI-TOF MS, ESI-QIT-reTOF MS, or ESI-oaTOF MS (LCT). Information such as the molecular weight, pI and peptide map of a protein can then be entered into a web-based protein database program such as MS-Fit (e.g., http://prospector.ucsf.edu) for protein identification.

### EXAMPLE 8

### Automated 3-D IE NP-RP-HPLC-ESI-oa TOF MS

This example describes an automated system for protein separation and identification based on charge, hydrophobicity, and mass. Protein samples are separated based on charge using an ion exchange (IE) column. Protein fractions are then trapped on a solid phase extraction (SPE) column for desalting using an automated Prospekt system. The Prospeckt system then directs the protein fractions to a nonporous-reverse phase HPLC column (NP-RP-HPLC). The samples are then identified using ESI oa TOF mass spectroscopy.

A. Protein Separation and Trapping by SPE

Siberian Permafrost whole cell lysate of sample 23-9-25 (obtained from Jim Tiendje, Department of Microbial Ecology, Michigan State University) was lysed using a chemical lysis procedure. The lysis buffer contained 6M guanidine-HCL, 20 mM n-octyl β-D-glucopyransoside and 50 mM Tris. The cells were vortexed vigorously and stored overnight at 0° C. The cells were then centrifuged at 17,000 rpm for 20 minutes. The supernatant was removed from the cellular material and then mixed 1:1 with an equilibration buffer for IE (10 mM KH₂PO₄, 5% MeOH, 0.1% n-octyl β-D glucopyranoside, pH 8). The sample was then injected into a Mini Q anion exchange column (Amersham Pharmacia, Uppsala, Sweden) with a flow rate of 1 ml/min at 27° C. Equilibration buffer was run through the column for 3 minutes, followed by a 0% to 100% gradient of buffer B (10 mM KH₂PO₄, 5% MeOH, 0.1% n-octyl β-D glucopyranoside, 1M NaCl, pH 7) in 15 minutes. A graph of the Mini Q column eluent is shown in FIG. 17.

Fractions (1 minute each) are each collected on a separate solid phase extraction (SPE) cartridge by directing the eluent from the IE through 10 C4 SPE cartridges. A Prospekt on-line automated SPE system (Spark Holland Instrumenten, The Netherlands) was utilized for the SPE, HPLC, and MS phases.

B. Protein Purification and Separation by NP-RP-HPLC

The initial mobile phase buffer for the RP analysis was 5% buffer B (0.1% TFA in ACN) in buffer A (0.1% TFA in H₂O). This solution was directed through the SPE cartridge until all the residual salt from the anion exchange mobile phase was removed. The eluent from the SPE cartridge was next directed by the Prospekt system directly to a HPLC for the second orthogonal separation phase.

Non Porous-RP columns (Eichrom Technologies, Darien, Ill.) were used as the second separation phase. A tandem column method was employed. ODSIIIE and ODSI NP RP HPLC columns (Eichrom Technoligies, Darien, Ill. contained 1.5 μm C18 (ODSI) non-porous silica beads. Column dimentions were 4.6*33 mm (ODSIIIE) and 4.6*14 mm (ODSI). The columns were maintained at 60° C. to improve selectivity. A flow rate of 0.5 mL/min at a pressure of 5000 psi was maintained. The columns were loaded, equilibrated in the initial buffer, and the gradient was started. A gradient of buffer B (0.1% TFA in ACN) was performed as follows: 5% B for 1.5 min, 5% B to 20% B in 2 min, 20% B to 35% B in 5 min, 35% B to 60% B in 15 min, 60% B to 100% B in 5 minutes. The eluent from the first HPLC column (ODSI) was directed into the second HPLC column (ODSIIIE).

Following the gradient, the initial mobile phase buffer was run through the RP column until a stable baseline is realized. The HPLC step was repeated for each of the SPE columns (each of which contained a 1 minute fraction from the anion exchange column).

C. Protein Identification by Mass Spectroscopy

Following separation by NP-RP-HPLC, protein fractions were analyzed online by MS to determine their molecular weight and abundance. Samples were analyzed by ESI oa TOF TIC (total ion count) mass spectroscopy. Mass spectroscopy conditions were as follows: capillary 2900V, sample cone 45V, extraction cone 3V, RF lens 1000V, desolution temp or 350° C., and source temp of 120° C.

Results of the ESI oa TOF TIC analysis are shown in FIGS. 18A and B. FIG. 18A shows the total ion profile of the fraction collected from 3 to 4 of the MiniQ column; FIG. 18B shows the total ion profile of the fraction collected from 7 to 8 minutes.

### EXAMPLE 9

### Differential Display Mapping

This example describes the separation of protein samples from normal and cancerous ovarian cell samples by IEF and NP RP HPLC, followed by detection with mass spectrometry and analysis with differential display.

A. Protein Separation by IEF

Proteins are extracted using a lysis buffer containing 6M Urea, 2M thiourea, 1.0% n-octyl-β-D-glucopyroanoside, 10 mM dithioerythritol (dTT) and 2.5% (w/v) carrier ampholytes (pI 3.5 to 10). After extraction the supematent protein is loaded into a Rotofor Isoelectric Focusing device. This device separates proteins in the liquid phase according to their isoelectric point (pI) The cell lysate is further diluted in an IEF running buffer containing 6M Urea, 2M thiourea, 0.5% n-octyl-β-D-glucopyranoside, 10 mM dTT and 2.5% w/v carrier ampholytes (pI 3.5 to 10.) The Rotofor is then run according to the standard procedure in Rotofor Manual (Biorad).

Alternatively, one of the following liquid-based IEF systems are used for the first dimension IEF separation:

1) Carrier Ampholyte Based Slab Gel IEF Separation with the Whole Gel Eluter (WGE).

In this case the protein solution is loaded onto a slab gel and the proteins separated into a series of gel-wide bands containing proteins of the same pI. These proteins are harvested using the Whole Gel Eluter (WGE, Biorad). Proteins are then isolated in liquid fractions that are ready for analysis by NPS RP HPLC. This type of gel can be loaded with up to 20 mg of protein.

2) IPG Slab Gel IEF Separation with the Whole Gel Eluter (WGE).

Here the proteins are loaded onto an Immoboline pI gradient slab gel and separated into series of gel-wide bands containing proteins of the same pI. These proteins are also harvested into liquid fractions that are ready for RP NPS HPLC. The IPG gel may be loaded with up to 60 mg of protein.

B. Protein Separation by NPS RP HPLC

Having obtained liquid fractions containing large amounts of pI-focused proteins, the second dimension separation is non-porous RP HPLC. Separations are performed at a flow rate of 0.4 mL per minute on an analytical (3.0×53 mm) NPS RP HPLC column containing 1.5 mm C18 (ODSI) non-porous beads (Eichrom Technologies.) The column is placed in a column heater (Timberline, Boulder, Colo.) and held at 65° C. The separations are performed using a water/acetonitrile gradient (0.1% TFA, 0.3% formic acid.) The gradient profile is as follows: 10-25% 2 mins, 25-35% 5 mins, 35-45% 10 mins, 45-75%, 10 mins, 75-100%, 1 min. Columns are packed with non-porous silica beads (Eichrom) to reduce problems of protein recovery associated with porous packings.

C. Protein Detection via Mass Spectrometry

The proteins that elute from the NPS RP HPLC separation must be analyzed by mass spectrometry to determine their molecular weight and identity. For this purpose the eluant from the HPLC column is split. One portion of the eluant is connected on-line to an Electrospray Ionization orthogonal acceleration Time of Flight Mass Spectrometer (ESI oa TOF-MS.) The ESI oaTOF/MS analyses are performed on an LCT equipped with a reflectron, 0.5 m flight tube and dual micro-channel plate detector. The source temperature is held at 120° C. and the desolvation temperature, 350° C. The nebulizer gas is held at 50% maximum flow, and the desolvation gas is held at 575 L/min. The capillary voltage is held at 2500 V, and the sample cone voltage is held at 35 V. The extraction cone is held a +3 V, and the RF lens is set to 1000 V. The RF DC offset for the first hexapole is +7 V and for the second hexapole, −2V. The detector is held at 3000 V. The pusher cycle time is set to 90 ms. The data is stored to an embedded pc at the rate of 1 Hz and then transferred to the main computer for generation of the data files and TIC.

Micromass' MassLynx v 3.4 and MaxEnt (version 1) software are used for data analysis. The TIC is scanned for regions that contained redundant multiply charged peaks, and those regions were combined for deconvolution. Deconvolution is performed using a target mass range of 5-85 KDa, 1 Da resolution, 0.75 Da peak width, and a 65% peak height value. The deconvoluted peaks are then combined into a single mass spectrum for each TIC. The combined mass spectrum is converted to a text file for input into the 2-D mapping software and the differential display software that were developed in-house.

The other portion of the HPLC eluant is split off to a Uv-Vis detector, followed by an auto collector where the proteins are collected in accordance with their peak profile from the UV-Vis detector. After collection the fractions are dried down to 50% of their original volume to remove the acetonitrile and TFA. To the reduced volume fractions 10% (v/v) 10 mM DTT, 10% (v/v) IM NH4HCO3 and 0.25 mg of TPCK-treated trypsin (Promega) is added. The fractions are then placed in a 37° C. warm room for 24 hrs. After 24 hrs, 2.5% (v/v) TFA is added to stop digestion and the fractions are stored at 4° C. until further analysis.

Prior to MALDI analysis, the proteins are purified and desalted using 2 mm C18 ZipTips (Millipore) with a final elution volume of 10 mL. 0.4 ml of this purified protein solution is spotted into a well on the MALDI plate and 0.4 ml of saturated a-CHCA (in 50% ACN, 1% TFA) is added on top of the sample before the sample dries. MALDI-MS is performed using a delayed extraction reflectron-equipped MALDI-TOF MS instrument (STR, Perseptive.) The repeller voltage is set at +25 kV, the grid voltage at 72% of repeller voltage, the delay time is 100 ns and the reflectron was set to a ratio of 1.12. 100-150 spectra are averaged for each peptide mass spectrum.

The peptide masses, along with the pI and molecular weight of the protein determined in previous parts of the experiment, are submitted to a database such as Ms-Fit for protein identification.

D. Differential Display

Differences between the two cell types are viewed as an image. A point by point subtraction for each data value at every m/z and pI value is taken. The image is prepared from that difference. Since differences can be either positive or negative, two colors are used. The specific color shows which cell type is more abundant and the color intensity indicates by how much. FIG. 23 shows the differential display plot of the 10-35 kDa region of a single pI range for two cell types. The 2-D map for the ES2 ovarian cancer cell line is on the left, and for normal ovarian epithelial cells, on the right. The differences between the two cells lines appear in the middle. The left plot shows a series of red bands, and the right plot shows a series of green bands. The middle plot shows some red and some green bands, as some proteins are more highly expressed in the cancer cell line, and other proteins are more highly expressed in the normal cells.

The horizontal X-axis of FIG. 23 is pI value and the vertical Y-axis is m/z ratio. A pI fraction spans several tenths of a pI unit over a range of 3 to 12 for a total of 20 fractions. The pI ranges of the fractions are not required to match between cell lines. Cell line A may contain fractions of A1 from pI 7.0 to 7.6, A2 from 7.6 to 8.0 and A3 from 8.0 to 9.0. Cell line B might span B 1 from 6.9 to 7.4, B2 from 7.4 to 8.1 and B3 from 8.1 to 8.8. In order to maintain a resolution of 0.1 pI in the difference display, the pI axis is further sub-divided into a least common fraction between the two cell lines, typically 0.1 pI unit. Thus, the data from one cell line fraction is used in more than one fraction of the difference display. The data from fraction A1 is used twice. Once for the difference with B1 over the 7.0 to 7.4 pI range, and again for the difference with B2 over the 7.4 to 7.6 pI range. Because there are many more resolution elements on the mass axis than pI axis, the image appears as bands contained within columns.

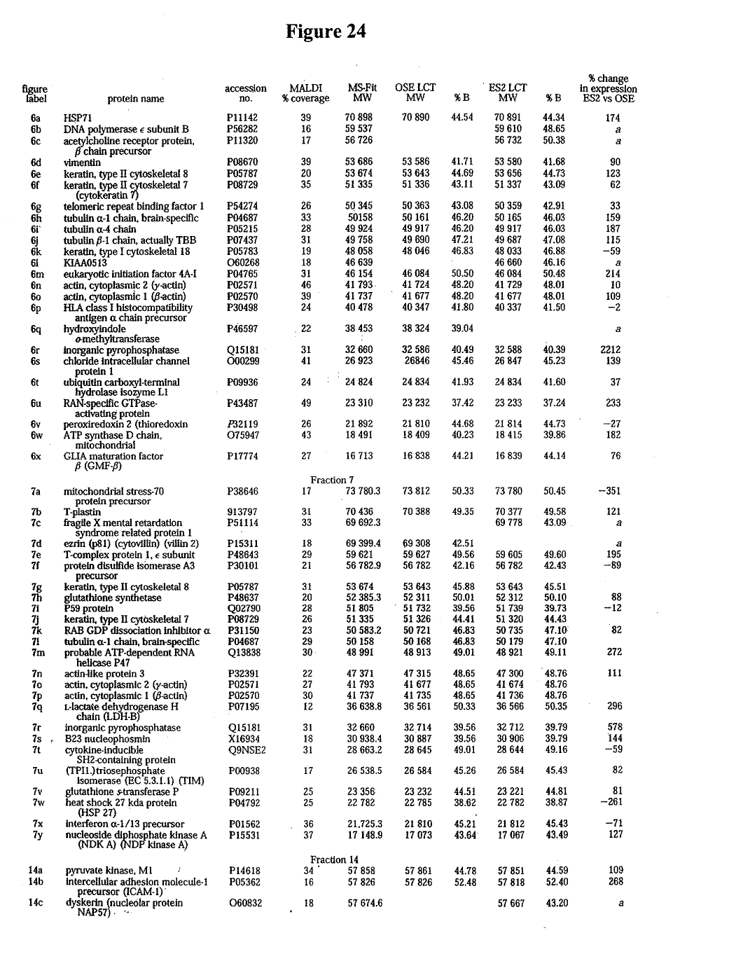

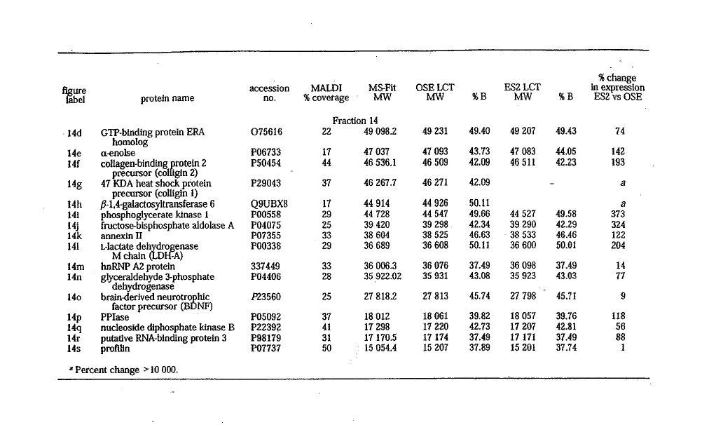

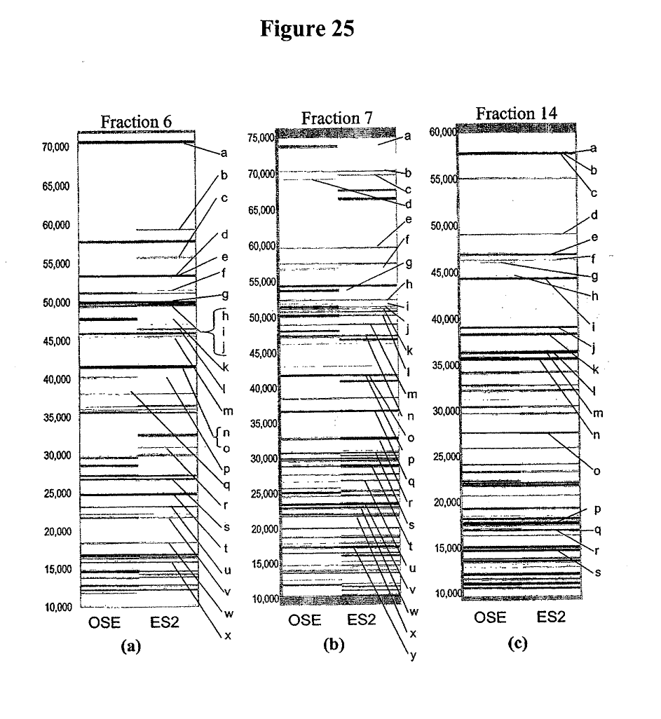

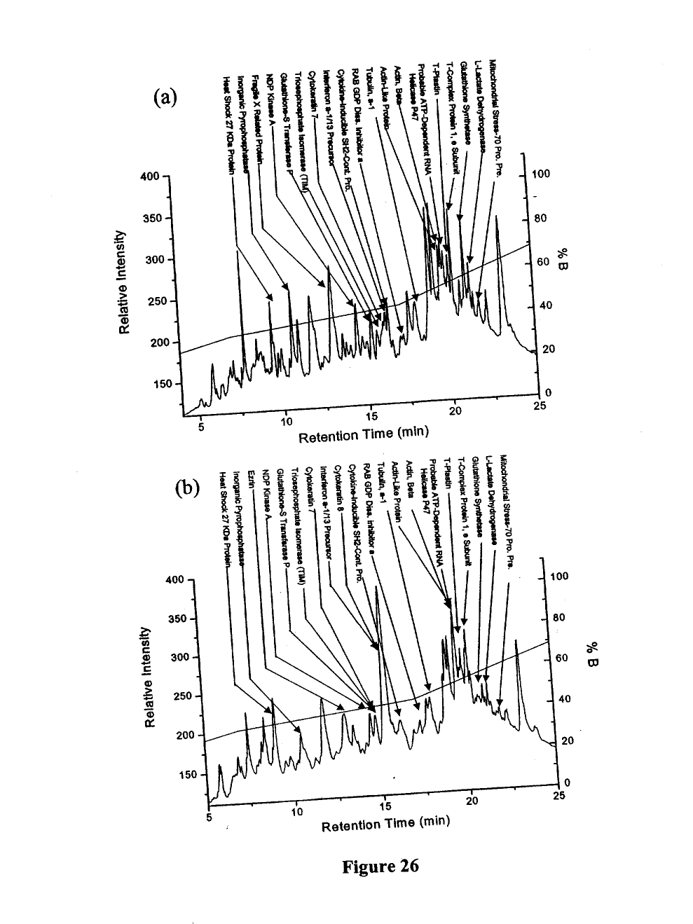

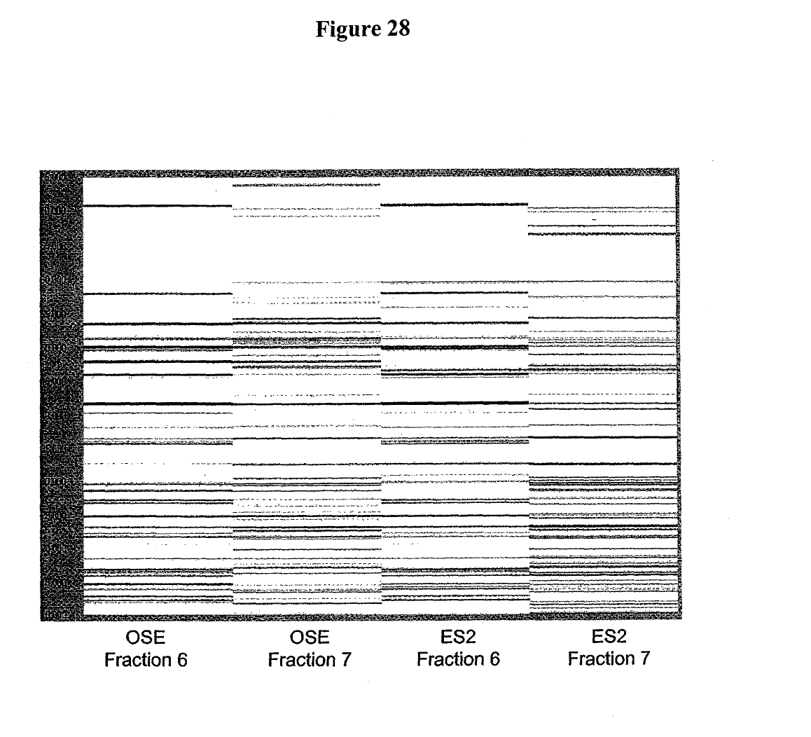

FIG. 24 shows a Table of proteins identified in ES2 and OSE with quantification and hydrophobicity comparison. FIG. 25 shows 2-Dimensional mass maps of MW versus pI comparing the ES2 cell line to the OSE cell line for Rotofor fraction nos. (a) 6, (b) 7, and (c) 14. The names of proteins identified by MALDI-TOFMS peptide mapping are listed with the corresponding MW bands according to the labeling scheme of FIG. 23. FIG. 26 shows NPS RP-HPLC chromatograms of Rotofor fraction 7 for FIG. 26(a) ES2 cell line and FIG. 26(b) OSE cell line with detection by UV absorption at 214 nm. The names of proteins identified by liquid fraction collection, tryptic digestion, and MALDI-TOFMS peptide mapping are listed with the corresponding chromatographic peak. FIG. 27 shows a Table of purported proteins not identified by MALDI but present in Fraction 6 in Both ES2 and OSE. FIG. 28 shows a comparison of the mass maps for fractions 6 and 7 between the OSE cell lines and the ES2 cell lines, demonstrating the limited overlap between the fractions.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim:

1. A method for summing mass spectrum data, comprising:
a) providing a mass spectrum generated from a separated protein sample;
b) identifying regions of said mass spectrum that contain mass data for a first protein; and
c) summing said regions of said mass spectrum to generate summed mass spectrum.

2. The method of claim 1, wherein said separated protein sample comprises a separated cell lysate.

3. The method of claim 2, wherein said separated cell lysate is separated in first and second separation dimensions.

4. The method of claim 3, wherein said first separation dimension represents protein isoelectric point and said second separation dimension represents protein hydrophobicity.

5. The method of claim 3, wherein said cell lysate is further separated based on molecular weight and abundance.

6. The method of claim 1, further comprising the step d) displaying said summed mass spectra.

7. The method of claim 6, wherein said summed mass spectra are displayed as a 2-dimensional map.

8. The method of claim 7, wherein said 2-dimensional map comprises a first axis representing isoelectric point and a second axis representing mass.

9. The method of claim 7, wherein said 2-dimensional map further displays protein abundance of proteins represented in said 2-dimensional map.

10. The method of claim 7, wherein proteins are represented as bands in said 2-dimensional map, and wherein the intensity of said bands represents relative protein abundance of said bands.

11. The method of claim 7, wherein said 2-dimensional map is displayed on a computer video screen.

12. The method of claim 1, wherein said summing of step c) is performed manually.

13. The method of claim 1, wherein said summing of step c) is performed by a computer processor.

14. A method for displaying proteins comprising:
a) providing:
i) a first 2-dimensional protein map representing a first sample comprising a plurality of proteins;
ii) a second 2-dimensional protein map representing a second sample comprising a plurality of proteins; and
iii) a computer system comprising display software, and a display screen; and
b) subtracting said second 2-dimensional protein map from said first 2-dimension protein map with said display software to generate a differential display map; and
c) displaying said differential display map on said display screen.

15. The method of claim 14, wherein said differential display map represents differences in protein composition between said first and second 2-dimensional protein maps as bands, and wherein each band represents one protein.

16. The method of claim 15, wherein said bands comprise bands of two different colors, and wherein each of said two different colors corresponds to proteins from each of said first and second samples.

17. The method of claim 15, wherein said bands comprise bands of two different color gradients, and wherein each of said two different color gradients correspond to proteins from each of said first and second samples.

18. The method of claim 15, wherein said differences in protein composition represent differences in abundance of the same protein displayed in each of said first and second 2-dimensional protein maps.

19. The method of claim 15, wherein said differences in protein composition represent the presence or absence proteins in each of said first and second 2-dimensional protein maps.

20. The method of claim 14, wherein said first and second 2-dimensional protein maps represent separation of said first and second proteins samples in a first dimension and a second dimension.

21. The method of claim 20, wherein said first dimension is isoelectric point and said second dimension is hydrophobicity.

22. The method of claim 20, wherein said first and second 2-dimensional protein maps further represent characterization of protein mass and abundance.

23. The method of claim 15, wherein said differential display map further comprises hyperlinks.

24. The method of claim 23, wherein said hyperlinks are links to information corresponding to proteins represented by said bands of said differential display map.

25. The method of claim 24, wherein said information is selected from the group consisting of protein identity, molecular weight, relative abundance, isolectric point, and hydrophobicity.

26. A system for displaying protein differential display maps, comprising:
a) a protein differential display map displayed on a display screen; and
b) a plurality of hyperlinks displayed on said display screen, wherein said hyperlinks correspond to individual regions of said protein differential display map, and wherein said hyperlinks are links to information corresponding to said regions.

27. The system of claim 26, wherein said protein differential display map represents differences in protein composition between first and second 2-dimensional protein maps.

28. The system of claim 27, wherein said differences in protein composition are represented as bands, and wherein each band represents one protein.

29. The system of claim 26, wherein each of said regions is a band corresponding to one protein.

30. The system of claim 29, wherein said information is selected from the group consisting of protein identity, molecular weight, relative abundance, isolectric point, and hydrophobicity.
